(19) United States
(12) Patent Application Publication
(10) Pub. No.: US 2025/0368650 A1
(43) Pub. Date: Dec. 4, 2025

(54) 5-(4-FLUOROPHENYL)-2,3-DIHYDRO-1H-IMIDAZO[1,2-A]IMIDAZOLE DERIVATIVES AS ALK INHIBITORS FOR THE TREATMENT OF FIBROSIS

(71) Applicant: CHIESI FARMACEUTICI S.P.A., Parma (IT)

(72) Inventors: Daniele PALA, Parma (IT); Daniela PIZZIRANI, Parma (IT); Sara GUARIENTO, Parma (IT); Paolo RONCHI, Parma (IT)

(73) Assignee: CHIESI FARMACEUTICI S.P.A., Parma (IT)

(21) Appl. No.: 18/876,818

(22) PCT Filed: Jun. 21, 2023
(86) PCT No.: PCT/EP2023/066728

§ 371 Date: Dec. 19, 2024

(30) Foreign Application Priority Data
Jun. 22, 2022 (EP) 22180542.7

(51) Int. Cl. C07D 487/04; A61K 31/4439; A61K 31/454; A61K 31/4545; A61K 31/496

(52) U.S. Cl. CPC C07D 487/04; A61K 31/4439; A61K 31/454; A61K 31/4545; A61K 31/496

(57) ABSTRACT

The present invention relates to 5-(4-fluorophenyl)-2,3-dihydro-1H-imidazo[1,2-a]imidazole derivatives of formula (I) as ALK5 inhibitors (transforming growth factor 3 (TGF3) type 1 receptor) for the treatment of fibrosis, such as e.g. pulmonary fibrosis, idiopathic pulmonary fibrosis (IPF), hepatic fibrosis, renal fibrosis, ocular fibrosis, cardiac fibrosis, arterial fibrosis and systemic sclerosis. An example with good activity is e.g. example 5: 2-(dimethylamino)-N-(2-fluoro-5-(6-(6-methylpyridin-2-yl)-2,3-dihydro-1H imidazo[1,2-a]imidazol-5-yl)phenyl)acetamide (Formula IA). Pharmacological data is provided: (Table 3)

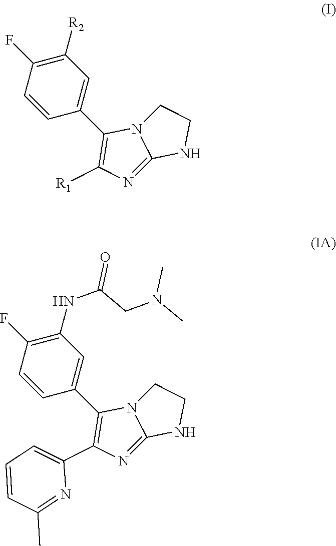

TABLE 3

| Example No | ALK5 pKᵢ |
|---|---|
| 2, 9, 10 | ≥9.0 |
| 1, 3, 4, 7, 8, 11, 12, 14 | 8.1-8.9 |
| 5, 6, 13 | 7.5-8.0 |

### FIELD OF THE INVENTION

The present invention relates to compounds inhibiting the transforming growth factor β (TGF β) type I receptor (ALK5) (hereinafter ALK5 inhibitors), methods of preparing such compounds, pharmaceutical compositions containing them and therapeutic use thereof. The compounds of the invention may be useful in the treatment of many diseases, disorders, or conditions associated with ALK5 signaling pathway.

### BACKGROUND OF THE INVENTION

The Transforming Growth Factor β (TGF β) is a protein belonging to the TGF β superfamily.

It is involved in several processes, both cellular, such as proliferation, migration and differentiation, and biological, including wound healing, immunosuppression, cancerogenesis and extracellular matrix production.

The TGF β superfamily also includes, among others, other members known as activins (Acts) (see e.g., Hinck A P, FEBS Letters 586 (2012); 1860-1870).

The binding of the peptide initiates the TGF β signaling cascade through the formation of a heterotetrameric complex composed of two different serine/threonine kinases receptors: type 1 (TGFβR1/ALK5) and type 2 (TGFβR2).

TGFβR1/ALK5 is recruited and activated through the phosphorylation of its intracellular domain by TGFβR2, leading in turn to the phosphorylation of the receptor-activated (R)-Smad family, resulting in the activation of target gene transcription (see e.g., Sheppard D., Proc Am Thorac Soc. (2006)(3); 413-417).

Similarly, to the TGF β signaling, the type I receptor for activin, ALK4, leads to the activation of target gene transcription (see e.g., Heldin C H et al., Cold Spring Harb Perspect Biol. (2016) August 1; 8(8)).

Several studies have linked an excessive and/or dysregulated TGFβ activity with many diseases including cancer and fibrosis (see e.g., Syed V, J Cell Biochem. (2016) June; 117(6):1279-87; Jakowlew S B. Cancer Metastasis Rev. (2006) September; 25(3):435-57). Among fibrotic disorders, a crucial role of TGFβ has been shown in organs such as lung, heart, liver, and kidney (see e.g., Alhamad E H, J Thorac Dis. (2015); 7(3):386-93). In particular, TGFβ expression is increased in fibrotic lung diseases, such as idiopathic pulmonary fibrosis (IPF), and in chronic inflammatory conditions, such as chronic obstructive pulmonary disease and asthma (see e.g., Thomas B J et al., Am J Respir Cell Mol Biol. (2016); (55):759-766).

In lung, TGFβ is expressed in several cell types, like epithelial cells, endothelial cells, connective tissue cells, macrophages and fibroblasts.

These cell populations may produce excess of TGFβ in IPF human lung tissue. Moreover, high levels of TGFβ have been detected in lung tissue and BAL of IPF patients (see e.g. Bergeron A et al., Eur Respir J (2003); 22:69-76).

TGFβ gene expression and TGFβ protein production have been observed to increase in a variety of animal models of pulmonary fibrosis caused by bleomycin, silica, asbestos, and radiation (see e.g. Wei F et al., Int Immunopharmacol. (2017) July; 48:67-75; Choe J Y et al., Inflamm Res. (2010) March; 59(3):177-88; Wang X et al., Respir Res (2009); 10, 36) and it has also been reported how the TGFβ expression is sufficient to induce progressive fibrosis in rodents (see e.g. Sime P J et al., J Clin Invest (1997); 100:768-776; Kim K K et al.).

Contrarily, TGFβ signaling inhibition obtained by employing knockout (KO) animals can inhibit fibrosis development through TGFβ-linked mechanisms (see e.g. Bonniaud P et al., Am J Respir Crit Care Med (2005); 171:889-898; 34).

Similar results have been achieved with inhibition of TGFβR1 in mouse bleomycin disease model (see e.g., Wei Y et al., J Clin Invest. (2017); 127(10):3675-3688).

Activin signalling dysregulation, similarly to TGFβ, is associated to fibroblasts proliferation, myofibroblasts differentiation and accumulation of extracellular matrix (ECM) (see e.g., Yamashita et al., J. Am. Soc. Nephrol. (2004) 15, 91-101). Moreover, overexpression of activin has been linked to pathological conditions and fibrosis development in different organs, such as liver (see e.g. Patella et al., Am. J. Physiol. Gastrointest. Liver Physiol. (2006) 290, G137-G144), kidney (see e.g., Agapova et al., Kidney Int. (2016) 89, 1231-1243), heart (see e.g., Yndestad et al., Circulation (2004) 109, 1379-1385), and lung (see e.g., de Kretser et al., Crit. Care (2013) 17:R263).

Taken together these data suggest the importance of targeting ALK5 receptor to treat pharmacologically the aforementioned diseases, linked to dysregulated TGF signaling pathway.

The TGFβ signaling is strongly involved in the cardiovascular homeostasis (see e.g., van Meeteren L A et al., Springer (2013)). Several studies in humans and mice have shown the main role of TGFβ in angiogenesis and vascular morphogenesis. Moreover, TGFβ plays a key role in the development and functionality of cardiac valves (see e.g. Liu A. C. et al., Am. J. Pathol. (2007), 171, 1407-1418). It is therefore clear the importance of a selective regulation of TGFβ pathway to target the pathological effects avoiding the suppression of the signaling needed for a correct homeostasis.

The answer to this crucial point could be addressed by using the inhalation route to deliver an antiTGFβ drug.

The inhalatory route would allow the treatment of the affected lung compartment bypassing the issue of the heart exposure.

Various compounds have been described in the literature as ALK5 and/or ALK4 inhibitors.

Imidazole derivatives have been disclosed in the literature as TGF-β inhibitors.

WO2020/123453 and WO2021/102468 (Theravance) disclose imidazole derivatives as ALK5 receptors useful for the treatment of, among other diseases, fibrosis.

WO2013/009140 (SK Chemicals Co) discloses 2-pyridyl substituted imidazole derivatives as ALK5 and/or ALK4 receptors useful for the treatment of, among others, renal-, liver- or pulmonary fibrosis.

WO2016/081364 (Rigel Pharmaceuticals Inc.) discloses imidazole derivatives as TGF-β inhibitors useful for the treatment of fibrotic disorders, such as involved in chronic renal disease and vascular disease.

WO2020/041562 (Clavius Pharmaceuticals LLC) discloses imidazole derivatives as TGF-β inhibitors, useful for the treatment of, among others, multiple sclerosis, idiopathic pulmonary fibrosis, Alzheimer's Disease and chronic kidney disease.

Of note, inhibition of ALK5 receptor may be useful for the treatment of fibrosis and diseases, disorders and conditions that result from fibrosis.

Several efforts have been done in the past years to develop novel ALK5 receptor inhibitors useful for the treatment of several diseases and some of those compounds have shown efficacy also in humans.

However, there remains a potential for developing inhibitors of receptors ALK5 characterized by good potency, useful for the treatment of diseases or conditions associated with a dysregulation of ALK5 signaling pathway, in particular fibrosis.

In particular, there remains a potential for developing inhibitors of receptor ALK5 useful for the treatment of diseases or conditions associated with a dysregulation of ALK5 signaling in the respiratory field, in particular idiopathic pulmonary fibrosis (IPF), to be administered by the inhalation route and characterized by a good inhalatory profile, that corresponds to a good activity in the lung, a good lung retention and to a low metabolic stability in order to minimize the systemic exposure and correlated safety issues.

In this direction, we have surprisingly found a new series of compounds of general formula (I) that solves the problem of providing potent inhibitors of ALK5 receptor for administration by inhalation, that shows, at the same time, remarkable selectivity over the kinome, a good inhalatory profile, high clearance, low systemic exposure, improved safety and tolerability.

### SUMMARY OF THE INVENTION

In a first aspect the present invention relates to compounds of formula (I)

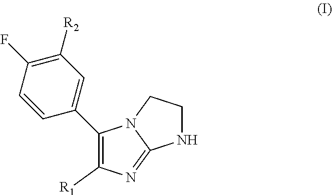

wherein
R₁ is selected from the group consisting of pyridyl optionally substituted by one or more —(C₁-C₆)alkyl, and phenyl optionally substituted by one or more halogen atoms;
R₂ is selected from the group consisting of —NH-heterocycloalkyl, wherein said heterocycloalkyl is optionally substituted by one or more —(C₁-C₆)alkyl; —NH—(C₁-C₆)alkylene-heterocycloalkyl, wherein said heterocycloalkyl is optionally substituted by one or more —(C₁-C₆)alkyl; —NH—C(O)—(C₁-C₆)alkylene-NRaRb; —NH—C(O)—(C₁-C₆)alkylene-heterocycloalkyl, wherein said heterocycloalkyl is optionally substituted by one or more groups selected from —(C₁-C₆)alkyl, —(C₁-C₆)haloalkyl and —(C₁-C₆)hydroxyalkyl;
Ra is —(C₁-C₆)alkyl;
Rb is —(C₁-C₆)alkyl;
and pharmaceutically acceptable salts thereof.

In a second aspect, the invention refers to a pharmaceutical composition comprising a compound of formula (I) and pharmaceutically acceptable salts thereof in admixture with one or more pharmaceutically acceptable carrier or excipient.

In a third aspect, the invention refers to a compound of formula (I) and pharmaceutically acceptable salts or to a pharmaceutical composition comprising a compound of formula (I) and pharmaceutically acceptable salts thereof for use as a medicament.

In a further aspect, the invention refers to a compound of formula (I) and pharmaceutically acceptable salts thereof or to a pharmaceutical composition comprising a compound of formula (I) and pharmaceutically acceptable salts thereof for use in preventing and/or treating a disease, disorder or condition mediated by ALK5 signaling pathway in a mammal.

In a further aspect, the invention refers to a compound of formula (I) and pharmaceutically acceptable salts thereof or to a pharmaceutical composition comprising a compound of formula (I) and pharmaceutically acceptable salts thereof for use in the prevention and/or treatment of fibrosis and/or diseases, disorders, or conditions that involve fibrosis.

In a further aspect, the invention refers to a compound of formula (I) and pharmaceutically acceptable salts thereof or to a pharmaceutical composition comprising a compound of formula (I) and pharmaceutically acceptable salts thereof for use in the prevention and/or treatment idiopathic pulmonary fibrosis (IPF).

### DETAILED DESCRIPTION OF THE INVENTION

### Definitions

Unless otherwise specified, the compound of formula (I) of the present invention is intended to include also tautomer or pharmaceutically acceptable salt or solvate thereof.

The term “pharmaceutically acceptable salts”, as used herein, refers to derivatives of compounds of formula (I) wherein the parent compound is suitably modified by converting any of the free acid or basic group, if present, into the corresponding addition salt with any base or acid conventionally intended as being pharmaceutically acceptable.

Suitable examples of said salts may thus include mineral or organic acid addition salts of basic residues such as amino groups, as well as mineral or organic basic addition salts of acid residues such as carboxylic groups.

Cations of inorganic bases which can be suitably used to prepare salts comprise ions of alkali or alkaline earth metals such as potassium, sodium, calcium or magnesium.

Those obtained by reacting the main compound, functioning as a base, with an inorganic or organic acid to form a salt comprise, for example, salts of hydrochloric acid, hydrobromic acid, sulfuric acid, phosphoric acid, methane sulfonic acid, camphor sulfonic acid, acetic acid, oxalic acid, maleic acid, fumaric acid, succinic acid and citric acid.

The term “halogen” or “halogen atoms” or “halo” as used herein includes fluorine, chlorine, bromine, and iodine atom.

The term “(Cₓ-C_{y})alkyl” wherein x and y are integers, refers to a straight or branched chain alkyl group having from x to y carbon atoms. Thus, when x is 1 and y is 6, for example, the term includes methyl, ethyl, n-propyl, isopropyl, n-butyl, isobutyl, sec-butyl, t-butyl, n-pentyl and n-hexyl.

The term “(Cₓ-C_{y})alkylene” wherein x and y are integers, refers to a Cₓ-C_{y}alkyl radical having in total two unsatisfied valencies, such as a divalent methylene radical.

The expressions “(Cₓ-C_{y})haloalkyl” wherein x and y are integers, refer to the above defined “Cₓ-C_{y}alkyl” groups wherein one or more hydrogen atoms are replaced by one or more halogen atoms, which can be the same or different. Examples of said “(Cₓ-C_{y})haloalkyl” groups may thus include halogenated, poly-halogenated and fully halogenated alkyl groups wherein all hydrogen atoms are replaced by halogen atoms, e.g. trifluoromethyl.

The term “(Cₓ-C_{y})cycloalkyl” wherein x and y are integers, refers to saturated cyclic hydrocarbon groups containing the indicated number of ring carbon atoms. Examples include cyclopropyl, cyclobutyl, cyclopentyl, cyclohexyl, cycloheptyl.

The term “aryl” refers to mono cyclic carbon ring systems which have 6 ring atoms wherein the ring is aromatic. Examples of suitable aryl monocyclic ring systems include, for instance, phenyl.

The term “heteroaryl” refers to a mono- or bi-cyclic aromatic group containing one or more heteroatoms selected from S, N and O, and includes groups having two such monocyclic rings, or one such monocyclic ring and one monocyclic aryl ring, which are fused through a common bond.

The term “(Cₓ-C_{y})heterocycloalkyl” wherein x and y are integers, refers to saturated or partially unsaturated monocyclic (Cₓ-C_{y})cycloalkyl groups in which at least one ring carbon atom is replaced by at least one heteroatom (e.g. N, S or O) or may bear an -oxo (═O) substituent group. Said heterocycloalkyl may be further optionally substituted on the available positions in the ring, namely on a carbon atom, or on a heteroatom available for substitution.

The term “(Cₓ-C_{y})hydroxyalkyl” wherein x and y are integers, refers to the above defined “(C₁-C₆)alkyl” groups wherein one or more hydrogen atoms are replaced by one or more hydroxy (OH) group.

Throughout the specification the use of an asterisk “*” in the definition of a structural formula, indicates the point of attachment for the radical group to the rest of the molecule.

A dash (“-”) that is not between two letters or symbols is meant to represent the point of attachment for a substituent.

The carbonyl group is herein preferably represented as —C(O)— as an alternative to the other common representations such as —CO—, —(CO)— or —C(═O)—.

In general, the bracketed group is a lateral group, not included into the chain, and brackets are used, when deemed useful, to help disambiguating linear chemical formulas.

The present invention relates to novel compounds differing from the structures disclosed in the art at least for a common new core scaffold. In fact, the invention relates to compounds that are imidazole derivatives, which are inhibitors of receptor ALK5, that have therapeutically desirable characteristics, particularly promising for some fibrosis, including idiopathic pulmonary fibrosis (IPF).

The compounds of the invention are active as inhibitors of ALK5 receptor, they are potent and show improved properties such as a remarkable selectivity over the kinome, a good inhalatory profile, high clearance, low systemic exposure, improved safety and tolerability.

In this respect, the state of the art does not describe or suggest imidazole derivatives of general formula (I) of the present invention having inhibitory activity on receptor ALK5 which represents a solution to the aforementioned need.

In more details, the present invention refers to a series of compounds represented by the general formula (I) as herein below described in details, which are endowed with an inhibitory activity on receptor ALK5.

Advantageously, the inhibitory action on receptor can be effective in the treatment of those diseases where these receptors play a relevant role in the pathogenesis such as fibrosis and disease, disorder and condition from fibrosis.

Differently from similar compounds of the prior art, the compounds of formula (I) of the present invention are able to act as inhibitors of ALK5 receptor, particularly appreciated by the skilled person when looking at a suitable and efficacious compound useful for the treatment of fibrosis, in particular idiopatic pulmonary fibrosis.

As indicated in the experimental part, in particular in Table 3, the compounds of formula (I) of the present invention show a notable potency with respect to their inhibitory activity on receptor ALK5, with pKi values greater than 7.5, confirming that they are able to inhibit ALK5 receptor involved in fibrosis and diseases that result from fibrosis.

Advantageously, the compounds of the present invention are endowed by a very high potency, they could be administered in human at a lower dosage respect to the compounds of the prior art, thus reducing the adverse events that typically occur administering higher dosages of drug.

In addition to being notably potent with respect to their inhibitory activity on receptor ALK5, the compounds of the present invention are also characterized by a good inhalatory profile, that permits to act effectively on the lung compartment and have, at the same time, a low metabolic stability, that allows to minimize the drawbacks associated with the systemic exposure, such as safety and tolerability issues.

Therefore, the compounds of the present invention are particularly appreciated by the skilled person when looking at a suitable and efficacious compounds useful for the treatment of fibrosis, in particular idiopathic pulmonary fibrosis, administered by the inhalation route and characterized by a good inhalatory profile, that corresponds to a good activity on the lung, a good lung retention and to a low metabolic stability, that minimizes the systemic exposure and correlated safety issues.

Thus, in one aspect the present invention relates to a compound of general formula (I)

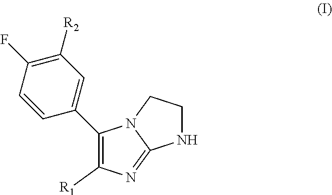

wherein
R₁ is selected from the group consisting of pyridyl optionally substituted by one or more —(C₁-C₆)alkyl, and phenyl optionally substituted by one or more halogen atoms;
R₂ is selected from the group consisting of —NH-heterocycloalkyl, wherein said heterocycloalkyl is optionally substituted by one or more —(C₁-C₆)alkyl; —NH—(C₁-C₆)alkylene-heterocycloalkyl, wherein said heterocycloalkyl is optionally substituted by one or more —(C₁-C₆)alkyl; —NH—C(O)—(C₁-C₆)alkylene-NRaRb; —NH—C(O)—(C₁-C₆)alkylene-heterocycloalkyl, wherein said heterocycloalkyl is optionally substituted by one or more groups selected from —(C₁-C₆)alkyl, —(C₁-C₆)haloalkyl and —(C₁-C₆)hydroxyalkyl;
Ra is —(C₁-C₆)alkyl;
Rb is —(C₁-C₆)alkyl;
and pharmaceutically acceptable salts thereof.

In a particularly preferred embodiment the present invention refers to a compound of formula (I), wherein R₁ is selected from the group consisting of pyridyl optionally substituted by one or more methyl, and phenyl optionally substituted by one or more halogen atoms selected from chlorine and fluorine, R₂ is selected from the group consisting of NH—C(O)—(C₁-C₆)alkylene-NRaRb and —NH—C(O)—(C₁-C₆)alkylene-heterocycloalkyl, wherein said heterocycloalkyl is optionally substituted by one or more groups selected from —(C₁-C₆)alkyl, —(C₁-C₆)haloalkyl and —(C₁-C₆)hydroxyalkyl;
and pharmaceutically acceptable salts thereof.

In a further preferred embodiment the present invention refers to a compound of formula (I), wherein R₁ is selected from the groups consisting of 6-methylpyridin-2-yl and -(5-chloro-2-fluorophenyl); and R₂ is selected from the group consisting of -3-(4-methylpiperazin-1-yl)propanamide, -3-(piperidin-1-yl)propanamide, -3-(4-(2-hydroxyethyl)piperazin-1-yl)propanamide, -3-(4-(2,2,2-trifluoroethyl)piperazin-1-yl)propanamide, 2-(dimethylamino)acetamide, -2-(4-methylpiperazin-1-yl)acetamide, -4-(4-methylpiperazin-1-yl)butanamide, 3-(dimethylamino)propanamide, -4-(piperidin-1-yl)butanamide and 1-(4-amino-4-oxobutyl)-1-methylpiperidin-1-ium.

According to a preferred embodiment, the invention refers to at least one of the compounds of Formula (I) listed in the Table 1 below and pharmaceutically acceptable salts thereof.

TABLE 1
List of preferred compounds of Formula (I)

| Example No. | Structure | Chemical Name |
|---|---|---|
| 1 | 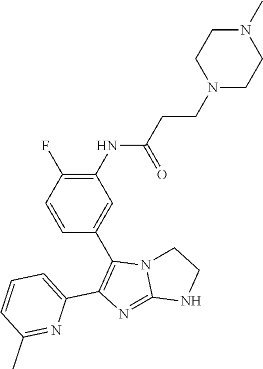 [SMILES: Cc1cccc(-c2nc3n(c2-c2ccc(F)c(NC(=O)CCN4CCN(C)CC4)c2)CCN3)n1] | N-(2-fluoro-5-(6-(6-methylpyridin-2- yl)-2,3-dihydro-1H-imidazo[1,2- a]imidazol-5-yl)phenyl)-3-(4- methylpiperazin-1-yl)propanamide |
| 2 | 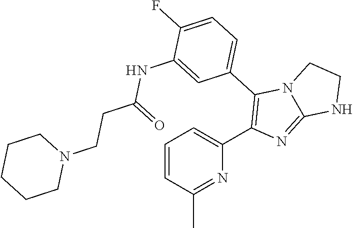 [SMILES: Cc1cccc(-c2nc3n(c2-c2ccc(F)c(NC(=O)CCN4CCCCC4)c2)CCN3)n1] | N-(2-fluoro-5-(6-(6-methylpyridin-2- yl)-2,3-dihydro-1H-imidazo[1,2- a]imidazol-5-yl)phenyl)-3-(piperidin-1- yl)propanamide |
| 3 | 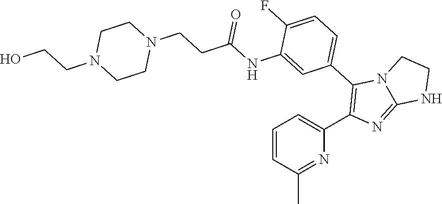 [SMILES: Cc1cccc(-c2nc3n(c2-c2ccc(F)c(NC(=O)CCN4CCN(CCO)CC4)c2)CCN3)n1] | N-(2-fluoro-5-(6-(6-methylpyridin-2- yl)-2,3-dihydro-1H-imidazo[1,2- a]imidazol-5-yl)phenyl)-3-(4-(2- hydroxyethyl)piperazin-1- yl)propanamide |
| 4 | 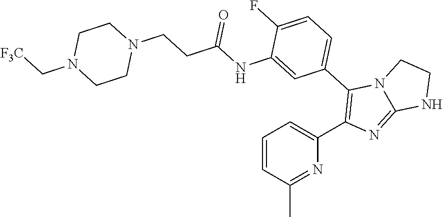 [SMILES: Cc1cccc(-c2nc3n(c2-c2ccc(F)c(NC(=O)CCN4CCN(CC(F)(F)F)CC4)c2)CCN3)n1] | N-(2-fluoro-5-(6-(6-methylpyridin-2- yl)-2,3-dihydro-1H-imidazo[1,2- a]imidazol-5-yl)phenyl)-3-(4-(2,2,2- trifluoroethyl)piperazin-1- yl)propanamide |
| 5 | 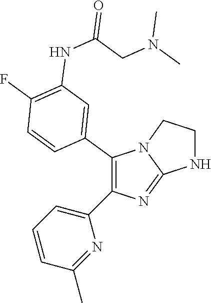 [SMILES: Cc1cccc(-c2nc3n(c2-c2ccc(F)c(NC(=O)CN(C)C)c2)CCN3)n1] | 2-(Dimethylamino)-N-(2-fluoro-5-(6- (6-methylpyridin-2-yl)-2,3-dihydro- 1H-imidazo[1,2-a]imidazol-5- yl)phenyl)acetamide |
| 6 | 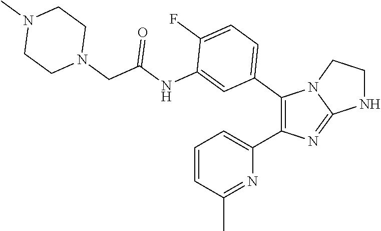 [SMILES: Cc1cccc(-c2nc3n(c2-c2ccc(F)c(NC(=O)CN4CCN(C)CC4)c2)CCN3)n1] | N-(2-fluoro-5-(6-(6-methylpyridin-2- yl)-2,3-dihydro-1H-imidazo[1,2- a]imidazol-5-yl)phenyl)-2-(4- methylpiperazin-1-yl)acetamide |
| 7 | 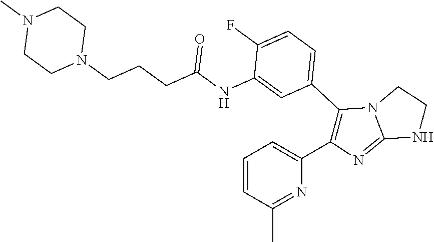 [SMILES: Cc1cccc(-c2nc3n(c2-c2ccc(F)c(NC(=O)CCCN4CCN(C)CC4)c2)CCN3)n1] | N-(2-fluoro-5-(6-(6-methylpyridin-2- yl)-2,3-dihydro-1H-imidazo[1,2- a]imidazol-5-yl)phenyl)-4-(4- methylpiperazin-1-yl)butanamide |
| 8 | 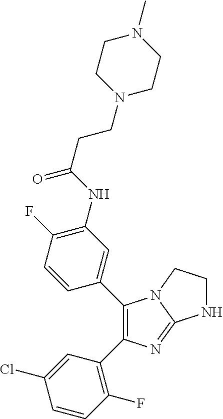 [SMILES: CN1CCN(CCC(=O)Nc2cc(-c3c(-c4cc(Cl)ccc4F)nc4n3CCN4)ccc2F)CC1] | N-(5-(6-(5-chloro-2-fluorophenyl)-2,3- dihydro-1H-imidazo[1,2-a]imidazol-5- yl)-2-fluorophenyl)-3-(4- methylpiperazin-1-yl)propanamide |
| 9 | 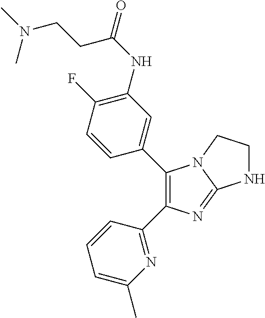 [SMILES: Cc1cccc(-c2nc3n(c2-c2ccc(F)c(NC(=O)CCN(C)C)c2)CCN3)n1] | 3-(Dimethylamino)-N-(2-fluoro-5-(6- (6-methylpyridin-2-yl)-2,3-dihydro- 1H-imidazo[1,2-a]imidazol-5- yl)phenyl)propanamide |
| 10 | 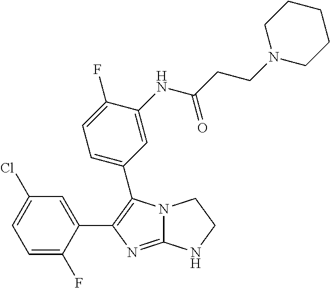 [SMILES: O=C(CCN1CCCCC1)Nc1cc(-c2c(-c3cc(Cl)ccc3F)nc3n2CCN3)ccc1F] | N-(5-(6-(5-chloro-2-fluorophenyl)-2,3- dihydro-1H-imidazo[1,2-a]imidazol-5- yl)-2-fluorophenyl)-3-(piperidin-1- yl)propanamide |
| 11 | 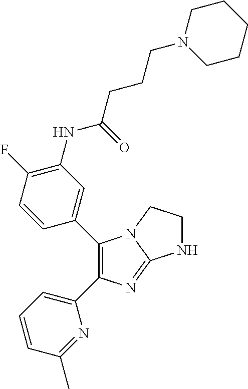 [SMILES: Cc1cccc(-c2nc3n(c2-c2ccc(F)c(NC(=O)CCCN4CCCCC4)c2)CCN3)n1] | N-(2-fluoro-5-(6-(6-methylpyridin-2- yl)-2,3-dihydro-1H-imidazo[1,2- a]imidazol-5-yl)phenyl)-4-(piperidin-1- yl)butanamide |
| 12 | 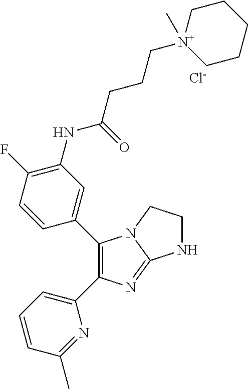 [SMILES: Cc1cccc(-c2nc3n(c2-c2ccc(F)c(NC(=O)CCC[N+]4(C)CCCCC4)c2)CCN3)n1 ; [Cl-]] | 1-(4-((2-Fluoro-5-(6-(6-methylpyridin- 2-yl)-2,3-dihydro-1H-imidazo[1,2- a]imidazol-5-yl)phenyl)amino)-4- oxobutyl)-1-methylpiperidin-1-ium chloride |

In another preferred embodiment the present invention refers to a compound of formula (I), wherein R₁ is pyridyl optionally substituted by one or more methyl, and R₂ is selected from the group consisting of NH-heterocycloalkyl, wherein said heterocycloalkyl is optionally substituted by one or more —(C₁-C₆)alkyl; —NH—(C₁-C₆)alkylene-heterocycloalkyl, wherein said heterocycloalkyl is optionally substituted by one or more —(C₁-C₆)alkyl; —NH—C(O)—(C₁-C₆)alkylene-heterocycloalkyl, wherein said heterocycloalkyl is optionally substituted by one or more —(C₁-C₆)alkyl;

and pharmaceutically acceptable salts thereof.

In a further preferred embodiment the present invention refers to a compound of formula (I), wherein R₁ is 6-methylpyridin-2-yl and R₂ is selected from the group consisting of -1-isopropylpiperidin-4-amine and -(1-methylpiperidin-4-yl)methanamine.

According to a preferred embodiment, the invention refers to at least one of the compounds of Formula (I) listed in the Table 2 below and pharmaceutically acceptable salts thereof.

TABLE 2
List of preferred compounds of Formula (I)

| Example No. | Structure | Chemical Name |
|---|---|---|
| 13 | 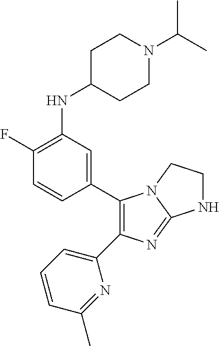 [SMILES: Cc1cccc(-c2nc3n(c2-c2ccc(F)c(NC4CCN(C(C)C)CC4)c2)CCN3)n1] | N-(2-fluoro-5-(6-(6-methylpyridin- 2-yl)-2,3-dihydro-1H-imidazo[1,2- a]imidazol-5-yl)phenyl)-1- isopropylpiperidin-4-amine |
| 14 | 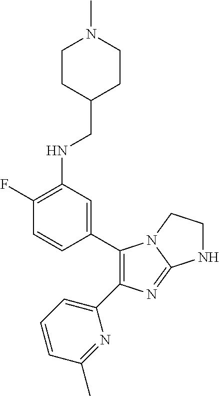 [SMILES: Cc1cccc(-c2nc3n(c2-c2ccc(F)c(NCC4CCN(C)CC4)c2)CCN3)n1] | 2-Fluoro-N-((1-methylpiperidin-4- yl)methyl)-5-(6-(6-methylpyridin-2- yl)-2,3-dihydro-1H-imidazo[1,2- a]imidazol-5-yl)aniline |

The compounds of formula (I) of the present invention have surprisingly been found to effectively inhibit the receptor ALK5. Advantageously, the inhibition of ALK5 may result in efficacious treatment of the diseases or condition wherein the ALK5 signaling is involved.

In this respect, it has now been found that the compounds of formula (I) of the present invention have an inhibitory drug potency, expressed as pIC₅₀ (negative logarithm of IC₅₀, half maximal inhibitory concentration) and subsequently converted to pKᵢ (negative logarithm of dissociate function Kᵢ), equal or higher than 7.5 on ALK5, as shown in the experimental part. Preferably, the compounds of the present invention have a pKᵢ on ALK5 between 8.1 and 8.9, more preferably higher than 9.0.

In one aspect, the present invention refers to a compound of formula (I) or a pharmaceutically acceptable salt thereof, for use as a medicament.

In a further aspect, the invention refers to the use of a compound of formula (I) of the invention, or a pharmaceutically acceptable salt thereof, in the manufacture of a medicament for the prevention and/or treatment of a disease, disorder or condition associated with dysregulated ALK5 signaling pathway.

In a preferred embodiment, the invention refers to a compound of formula (I) or a pharmaceutically acceptable salt thereof, for use in the prevention and/or treatment of a disease, disorder or condition associated with dysregulated ALK5 signaling pathway. In one embodiment, the present invention refers to a compound of formula (I) useful for the prevention and/or treatment of fibrosis and/or diseases, disorders, or conditions that involve fibrosis.

The invention also provides a method for the prevention and/or treatment of a disease, disorder or condition associated with dysregulated ALK5 signaling pathway, said method comprising administering to a patient in need of such treatment a therapeutically effective amount of a compound of the invention.

In particular the invention refers to a method for the prevention and/or treatment of fibrosis and/or diseases, disorders, or conditions that involve fibrosis, wherein said method comprises the administration of a proper amount of a compound of formula (I) to a patient in the need thereof.

The terms “fibrosis” or “fibrosing disorder,” as used herein, refers to conditions that are associated with the abnormal accumulation of cells and/or fibronectin and/or collagen and/or increased fibroblast recruitment and include but are not limited to fibrosis of individual organs or tissues such as the heart, kidney, liver, joints, lung, pleural tissue, peritoneal tissue, skin, cornea, retina, musculoskeletal and digestive tract. Preferably, the compounds of formula (I) of the present invention, or a pharmaceutical composition comprising a compound of formula (I), are useful for the treatment and/or prevention of fibrosis such as pulmonary fibrosis, idiopathic pulmonary fibrosis (IPF), hepatic fibrosis, renal fibrosis, ocular fibrosis, cardiac fibrosis, arterial fibrosis and systemic sclerosis.

More preferably, the compounds of formula (I) of the present invention, or a pharmaceutical composition comprising a compound of formula (I), are useful for the treatment of idiopathic pulmonary fibrosis (IPF).

The methods of treatment of the invention comprise administering a safe and effective amount of a compound of formula (I) or a pharmaceutically acceptable salt thereof to a patient in need thereof. As used herein, “safe and effective amount” in reference to a compound of formula (I) or a pharmaceutically acceptable salt thereof or other pharmaceutically-active agent means an amount of the compound sufficient to treat the patient's condition but low enough to avoid serious side effects and it can nevertheless be routinely determined by the skilled artisan. The compounds of formula (I) or pharmaceutically acceptable salts thereof may be administered once or according to a dosing regimen wherein a number of doses are administered at varying intervals of time for a given period of time. Typical daily dosages may vary depending upon the particular route of administration chosen.

In one embodiment, the invention refers to a pharmaceutical composition of compounds of formula (I) in admixture with one or more pharmaceutically acceptable carrier or excipient, for example those described in Remington's Pharmaceutical Sciences Handbook, XVII Ed., Mack Pub., N.Y., U.S.A.

Administration of the compounds of the invention and their pharmaceutical compositions may be accomplished according to patient needs, for example, orally, nasally, parenterally (subcutaneously, intravenously, intramuscularly, intrasternally and by infusion) and by inhalation.

Preferably, the compounds of the present invention are administered orally or by inhalation. More preferably, the compounds of the present invention are administered by inhalation.

In one preferred embodiment, the pharmaceutical composition comprising the compound of formula (I) is a solid oral dosage form such as tablets, gelcaps, capsules, caplets, granules, lozenges and bulk powders. The compounds of the invention can be administered alone or combined with various pharmaceutically acceptable carriers, diluents (such as sucrose, mannitol, lactose, starches) and known excipients, including suspending agents, solubilizers, buffering agents, binders, disintegrants, preservatives, colorants, flavorants, lubricants and the like.

In one embodiment, the pharmaceutical composition comprising the compound of formula (I) is a tablet.

In a further embodiment, the pharmaceutical composition comprising a compound of formula (I) is a liquid oral dosage forms such as aqueous and non-aqueous solutions, emulsions, suspensions, syrups, and elixirs. Such liquid dosage forms can also contain suitable known inert diluents such as water and suitable known excipients such as preservatives, wetting agents, sweeteners, flavorants, as well as agents for emulsifying and/or suspending the compounds of the invention. The compounds of the invention may be injected, for example, intravenously, in the form of an isotonic sterile solution.

In a further embodiment, the pharmaceutical composition comprising the compound of formula (I) is an inhalable preparation such as inhalable powders, propellant-containing metering aerosols or propellant-free inhalable formulations.

For administration as a dry powder, single- or multi-dose inhalers known from the prior art may be utilized. In that case the powder may be filled in gelatine, plastic or other capsules, cartridges or blister packs or in a reservoir.

A diluent or carrier chemically inert to the compounds of the invention, e.g. lactose or any other additive suitable for improving the respirable fraction may be added to the powdered compounds of the invention.

Inhalation aerosols containing propellant gas such as hydrofluoroalkanes may contain the compounds of the invention either in solution or in dispersed form. The propellant-driven formulations may also contain other ingredients such as co-solvents, stabilizers and optionally other excipients.

The propellant-free inhalable formulations comprising the compounds of the invention may be in form of solutions or suspensions in an aqueous, alcoholic or hydroalcoholic medium and they may be delivered by jet or ultrasonic nebulizers known from the prior art or by soft-mist nebulizers.

The compounds of the invention are administered as the sole active agent or in combination with other pharmaceutical active ingredients.

The dosages of the compounds of the invention depend upon a variety of factors including among others the particular disease to be treated, the severity of the symptoms, the route of administration and the like.

The invention is also directed to a device comprising a pharmaceutical composition comprising a compound of formula (I) according to the invention, in form of a single- or multi-dose dry powder inhaler or a metered dose inhaler.

All preferred groups or embodiments described above for compounds of formula (I) may be combined among each other and apply as well mutatis mutandis.

The various aspects of the invention described in this application are illustrated by the following examples which are not meant to limit the invention in any way.

The compounds of the invention, including all the compounds here above listed, can be prepared from readily available starting materials using the following general methods and procedures or by using slightly modified processes readily available to those of ordinary skill in the art. Although a particular embodiment of the present invention may be shown or described herein, those skilled in the art will recognize that all embodiments or aspects of the present invention can be obtained using the methods described herein or by using other known methods, reagents and starting materials. When typical or preferred process conditions (i.e. reaction temperatures, times, mole ratios of reactants, solvents, pressures, etc.) are given, other process conditions can also be used unless otherwise stated. While the optimum reaction conditions may vary depending on the particular reactants or solvent used, such conditions can be readily determined by those skilled in the art by routine optimization procedures.

Thus, processes described below and reported in the following schemes should not be viewed as limiting the scope of the synthetic methods available for the preparation of the compounds of the invention.

The compounds of formula (I) including all the compounds or at least one of the here above listed can be generally prepared according to the procedures outlined in detail in the Schemes shown below, using generally known methods.

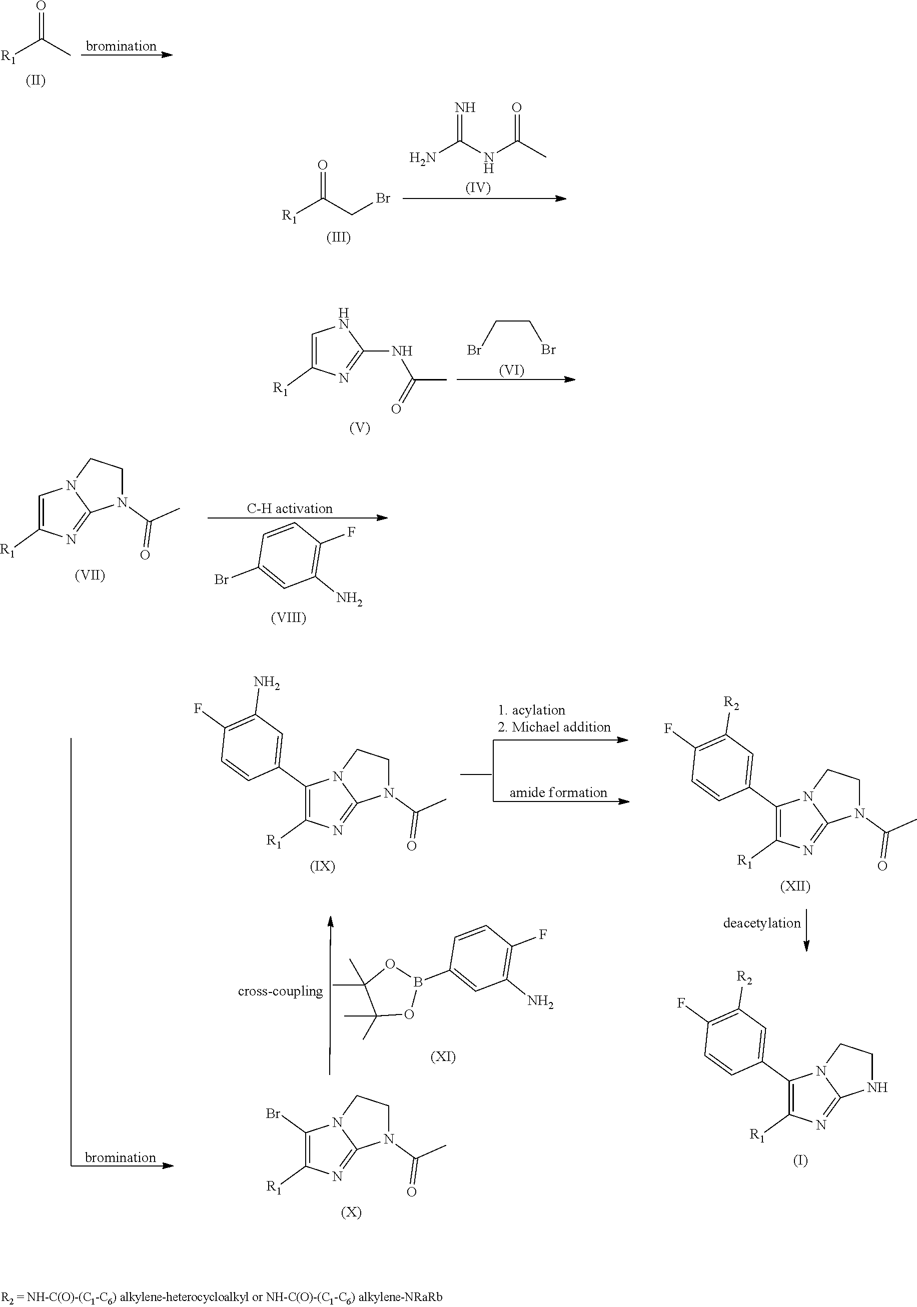

In one embodiment of the present invention, compounds of formula (I), wherein R₁ is defined as above and R₂ is selected from the groups —NH—C(O)—(C₁-C₆)alkylene-heterocycloalkyl or —NH—C(O)—(C₁-C₆)alkylene-NRaRb, may be prepared as described in Scheme 1, starting from commercially available compound (II), wherein R₁ is defined as above. Compound (III) may be prepared by bromination of compound (II). The reagents suitable for this reaction are, but not limited to, bromine and hydrogen bromide in acetic acid. Compounds of formula (V) may be obtained by cyclization reaction between compound (III) and commercially available compound (IV). Cyclization conditions include a suitable base, such as K₂CO₃, in a polar aprotic solvent, as for example MeCN and the like, and an appropriate temperature, for example, 50° C.

Commercially available compound (VI) can be employed in a cyclization reaction with compounds (V) to yield compounds of formula (VII). Cyclization conditions comprise the use of a proper base, such as for example K₂CO₃, a suitable solvent as MeCN and an appropriate temperature as, for instance, 80° C.

A compound of formula (VII) can undergo a metal-catalyzed C-H activation coupling with suitable halides of formula (VIII) to give compounds (IX). Typical reaction conditions include the use of palladium catalyst, such as Pd₂(dba)₃ or Pd(OAc)₂, a proper ligand as, for example, tri-o-tolylphosphane and a suitable base, as for example K₂CO₃ in a proper solvent, such as N,N-dimethylacetamide or N-methyl pyrrolidone, and an appropriate temperature, as 140° C.

In some cases, compounds (IX) can be prepared by bromination of compound (VII) with a suitable reagent such as, for example, N-bromo succinimide, followed by metal-catalyzed cross-coupling reaction of the obtained compounds of formula (X). Typical cross-coupling reaction may be Suzuki coupling, or similar as described in “Transition Metals for 15 Organic Synthesis”, 2nd Ed, 1, 2004. Representative Suzuki reaction conditions include reacting compound (X) with appropriate boronic acid (XI), in the presence of a palladium catalyst, such as Pd(dppf)Cl₂ or Pd(Ph₃P)₄, a suitable base, as for example Na₂CO₃ or K₂CO₃, in a mixture of solvents, such as 1,4-dioxane and water, at an appropriate temperature such as 100° C.

A compound of formula (XII), wherein R₂ is selected from the groups consisting of —NH—C(O)—(C₁-C₆)alkylene-heterocycloalkyl or —NH—C(O)—(C₁-C₆)alkylene-NRaRb, can be obtained from compound (IX) via a two-step preparation. First, compound (IX) can be reacted with suitable acylating agents, such as for example 3-bromopropionyl chloride, in the presence of proper organic base, as triethylamine or similar, and in a non-polar aprotic solvent as, for example, DCM, at an appropriate temperature, such as 0° C. or below. The resulting α,β-unsaturated carbonyl compound can promptly undergo 1,4-Michael addition in the presence of suitable nucleophiles, such as aliphatic amines, to give compounds of formula (XII).

In some cases, compounds (XII) can be prepared by amide coupling of compounds (IX) with suitable acid derivatives in the presence of proper coupling reagent, as for instance HATU or EDC, a base, such as triethylamine or diisopropylethylamine, in a solvent as DCM or DMF, at room temperature.

Finally, a compound of formula (I) can be obtained by treating compounds (XII) under acidic or basic conditions, using for example hydrochloric acid or sodium hydroxide, respectively, in polar protic solvent, such as methanol or ethanol, at an appropriate temperature, as 60° C.

In another embodiment of the present invention, wherein R₁ is defined as above and R₂ is selected from the groups —NH-heterocycloalkyl or —NH—(C₁-C₆)alkylene-heterocycloalkyl, compounds of formula (I) can be prepared as described in Scheme 2, using generally known methods.

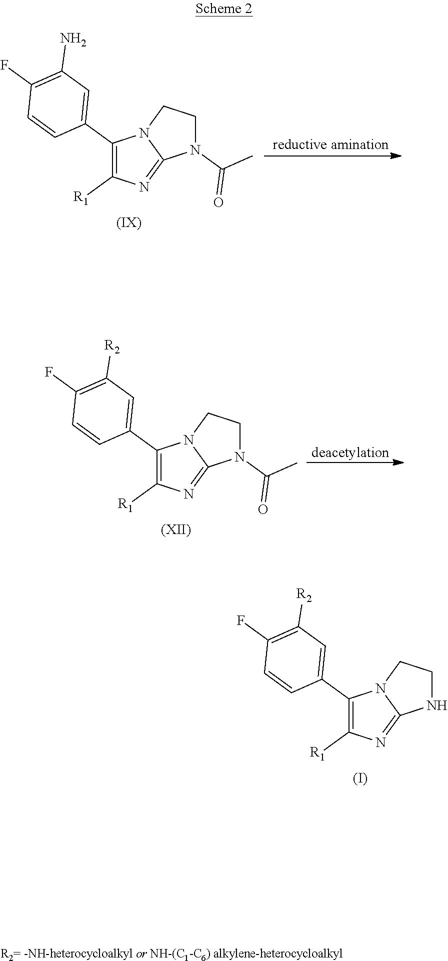

Compounds of formula (IX), prepared as described in Scheme 1, can react with suitable carbonyl compounds, such as aldehydes and ketons, under reductive amination conditions to give compounds (XII), wherein R₂ is —NH-heterocycloalkyl or —NH—(C₁-C₆)alkylene-heterocycloalkyl. Typical reductive amination conditions comprise the use of a reducing agent, such as sodium cyanoborohydride, sodium triacetoxyborohydride and the like, under mild acidic conditions, typically in polar or apolar aprotic solvents, as for instance, THF or DCM.

Final cleavage of the acetyl groups of compounds (XII), wherein R₁ is defined as above and R₂ is —NH-heterocycloalkyl or —NH—(C₁-C₆)alkylene-heterocycloalkyl, under mild acidic or basic conditions as previously described, may lead to compounds of formula (I).

### PREPARATIONS OF INTERMEDIATES AND EXAMPLES

Chemical Names of the compounds were generated with ChemDraw Version 19.1.1.21.

All reagents, for which the synthesis is not described in the experimental part, are either commercially available, or are known compounds or may be formed from known compounds by known methods by a person skilled in the art.

### Abbreviation—Meaning

AcOH=acetic acid; Aq.=aqueous; Ar=argon; BBr₃=boron tribromide; DCM=dichloromethane; DMA=dimethylacetamide; DMAP=4-(dimethylamino)pyridine; DMF=dimethylformamide; DMSO=dimethylsulfoxide; EDC·HCl=1-ethyl-3-(3-dimethylaminopropyl)carbodiimide; EtOAc=ethyl acetate; EtOH=ethanol; HATU=1-[bis(dimethylamino)methylene]-1H-1,2,3-triazolo[4,5-b]pyridinium 3-oxid hexafluorophosphate; HBr=hydrobromic acid; HCl=hydrochloric acid; HCOOH=formic acid; HPLC=high performance liquid chromatography; K₂CO₃=potassium carbonate; KOAc=potassium acetate; LC-MS=liquid chromatography mass spectrometry; MeCN=acetonitrile; MeOH=methanol; N₂=nitrogen; NaHCO₃=sodium bicarbonate; NaOH=sodium hydroxide; Na₂SO₄=sodium sulfate; NH-silica=amino-functionalized silica; NBS=N-bromo succinimmide; NMP=N-methyl-2-pyrrolidone; NMR=nuclear magnetic resonance; Pd₂(dba)₃=tris(dibenzylideneacetone)dipalladium(0); Pd(dppf)Cl₂ DCM=[1,1′-bis(diphenylphosphino)ferrocene]dichloropalladium(II), complex with dichloromethane;
Pd(OAc)₂=palladium(II) acetate; Pd(PPh₃)₄=tetrakis(triphenylphosphine)palladium(0);
pTLC=preparative thin layer chromatography; RP=reverse phase; rt=retention time; RT=room temperature; Sat.=saturated; SCX=strong cation exchange; STAB=sodium triacetoxyborohydride; TEA=triethylamine; THF=tetrahydrofuran; UPLC-MS=ultra-performance liquid chromatography mass spectrometry

### General Experimental Details and Methods

### Analytical Method

### Instruments, Materials and Methods Employed for Analyses:

### NMR

¹H Nuclear magnetic resonance (NMR) spectroscopy was carried out using a Bruker or Varian instruments operating at 300 or 400 MHz using the stated solvent at around room temperature unless otherwise specified. In all cases, NMR data were consistent with the proposed structures. Characteristic chemical shifts (δ) are given in parts-per-million using conventional abbreviations for designation of major peaks: e.g. s, singlet; d, doublet; t, triplet; q, quartet; dd, doublet of doublets; dt, doublet of triplets; m, multiplet; br, broad. Coupling constants (J values) are given in hertz (Hz).

### LC/UV/MS Analytical Methods

Materials: HCCOH ≥98% (Sigma-Aldrich), MeCN for HPLC UV/gradient grade (Baker), μQ-water for LCMS.

UPLC-MS measurements were performed on Waters ACQUITY UPLC I-Class PLUS System with Waters SQ Detector 2 (ESI-MS, capillary voltage: 3000 V, cone voltage: 40 V, de-solvation gas: 1000 L/h, de-solvation T: 500° C.), equipped with Acquity UPLC BEH C18 1.7 μm (2.1×100 mm), column no. 186002352, using 20-100% MeCN in water gradient with 0.1% HCOOH (flow: 0.5 mL/min).

### LCMS Methods:

LCMS measurements (for Methods A-C) were performed on Dionex UHPLC Ultimate 3000 with DAD detector/Thermo Scientific MSQ Plus system, equipped with Kinetex® 2.6 μm XB-C18 (4.6×50 mm), 110A, column no. OOB-4496-E0. Detection range: 190-350 nm±4 nm. Flow: 1.0 mL/min. Column temperature: 25° C. Autosampler temperature: 20° C.

LCMS measurements (for Method D) were performed on Aquity UPLC-QDa Mass Spectrometer with a C18-reverse-phase column (50×2.1 mm Acquity CSH with 1.7 m particle size). Flow: 1.0 mL/min. Column temperature: 40° C. Elution with A: 95/5 water/MeCN+0.05% HCOOH; B: 95/5 MeCN/water+0.05% HCOOH. Detection-MS, UV PDA. MS ionisation method-Electrospray (positive/negative ion).

LCMS measurements (for Method E) were performed on UPLC/PDA/MS Acquity™ system coupled with Micromass ZQ™ with Acquity Kinetex 1.7 μm EVO C18 100A, 2.1×50 mm. Flow: 1.0 mL/min. Column temperature: 40° C. Elution with A: 10 mM aqueous solution of NH₄HCO₃ adjusted to pH=10 with ammonia; B: MeCN. The UV detection range was 210-350 nm and ES+/ES− range was 100 to 1500 AMU.

### Method A:

Analysis time: 7 min. Mobile phase A: 0.1% v/v MQ water solution of HCOOH. Mobile phase B: 0.1% v/v MeCN solution of HCOOH. Elution gradient: 0.00-1.00 min (95% A, 5% B), 4.75-5.25 min (20% A, 80% B), 6.00-7.00 min (95% A, 5% B).

### Method B:

Analysis time: 6 min. Mobile phase A: 0.1% v/v MQ water solution of HCOOH. Mobile phase B: 0.1% v/v MeCN solution of HCOOH. Elution gradient: 0.00 min (90% A, 10% B), 3.35-3.75 min (30% A, 70% B), 3.90-4.75 min (5% A, 95% B), 5.00-6.00 min (90% A, 10% B).

### Method C:

Analysis time: 7 min. Mobile phase A: 0.1% v/v MQ water solution of HCOOH. Mobile phase B: 0.1% v/v MeCN solution of HCOOH. Elution gradient: 0.00 min (100% A, 0% B), 1.00 min (95% A, 5% B), 4.00 min (80% A, 20% B), 4.75-5.25 min (20% A, 80% B), 6.00 min (95% A, 5% B), 7.00 (100% A, 0% B).

### Method D:

Analysis time: 2 min. Mobile phase A: 95/5 water/MeCN+0.05% HCOOH; B: 95/5 MeCN/water+0.05% HCOOH. Elution gradient: 0.00 min (99% A, 1% B), 1.50 min (0.1% A, 99.9% B), 1.90 min (0.1% A, 99.9% B), 2.00 min (99.0% A, 1% B).

### Method E:

Analysis time: 2 min. Mobile phase A: 10 mM aqueous solution of NH₄HCO₃ adjusted to pH=10 with ammonia; B: MeCN. Elution gradient: 0.00 min (97% A, 3% B), 1.50 min (0.1% A, 99.9% B), 1.90 min (0.1% A, 99.9% B), 2.00 min (97.0% A, 3% B)

### General Synthetic Procedures

### Procedure A

To a 0.1M solution of the proper Intermediate (1.0 mmol) in DMA or NMP, suitable bromo derivative (1.5 mmol) was added, followed by K₂CO₃ (2.0 eq), tri-o-tolylphosphane (0.1 mmol) and Pd(OAc)₂ (0.05 mmol). The reaction mixture was heated to 140° C. and stirred for 2-16 h. Proper purification method yielded the desired product.

### Procedure B

To a solution of the suitable Intermediate (1.0 mmol) in a 3:1 mixture of 1,4-dioxane (3.0 mL)/water (1.0 mL), proper boronic ester or acid (1.2 mmol), K₂CO₃ (3.0 mmol) and Pd(PPh₃)₄ (0.15 mmol) were added. The mixture was heated to 100° C. and stirred for 2-16 h. The reaction was diluted with DCM and quenched with water. The two phases were separated and the organic layer was filtered through a phase separator tube. Volatiles were removed under reduced pressure. Proper purification method yielded the desired product.

### Procedure C

Proper Intermediate (1.0 mmol) was dissolved in EtOH (0.1M) or MeCN (0.1M) and aqueous HCl (2.0M, 5.0 to 10 mmol) was added. The resulting mixture was heated to 60° C. and stirred for 3-12 h. Volatiles were removed under reduced pressure. Proper purification yielded the desired product.

### Procedure D

Proper Intermediate (1.0 mmol) was dissolved in EtOH (0.1M) or MeOH (0.1M) and aqueous NaOH (1.0M, 1.5 to 5.0 mmol) was added. The reaction mixture was stirred at RT or heated up to 60° C. for 3-12 h. Volatiles were removed under reduced pressure. Proper purification yielded the desired product.

### PREPARATIONS OF INTERMEDIATES

### Intermediate 1: 2-Bromo-1-(6-methylpyridin-2-yl)ethan-1-one

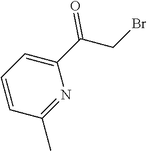

1-(6-Methylpyridin-2-yl)ethan-1-one (50 g, 370 mmol) was dissolved in HBr solution in AcOH 33% (100 mL). Then, bromine (21 mL, 407.4 mmol) was added dropwise to the solution over 30 min. The reaction was stirred for 6 h at RT, then diluted with DCM (500 mL) and filtered. The dried solid was suspended in DCM/water mixture (1:1 v/v) (1.5 L) and neutralized with solid NaHCO₃. The two phases were separated and the aqueous layer extracted with DCM. Combined organics were dried over Na₂SO₄, filtered and volatiles were removed under reduced pressure to yield the title compound (36.7 g, 172.0 mmol, 46% yield).

UPLC-MS (254 nm): m/z (M+1) 214, rt=1.68 min.

### Intermediate 2: 2-Bromo-1-(5-chloro-2-fluorophenyl)ethan-1-one

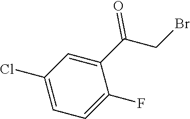 [SMILES: O=C(CBr)c1cc(Cl)ccc1F]

Prepared from 1-(5-chloro-2-fluorophenyl)ethan-1-one (0.500 g, 2.90 mmol) following a similar procedure as for Intermediate 1. Purification by flash chromatography (gradient of elution from 0% to 50% of EtOAc in heptane) yielded the title compound (0.546 g, 2.17 mmol, 75% yield).

LC-MS (ESI): m/z (M+1) no ionization, rt=1.09 min (Method D).

### Intermediate 3: N-(4-(6-Methylpyridin-2-yl)-1H-imidazol-2-yl)acetamide

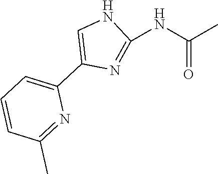 [SMILES: CC(=O)Nc1nc(-c2cccc(C)n2)c[nH]1]

To a suspension of Intermediate 1 (50.4 g, 253.3 mmol) in MeCN (50 mL), N-acetylguanidine (30.7 g, 304.0 mmol) and K₂CO₃ (42.0 g, 304.0 mmol) were added. The reaction mixture was stirred at 50° C. for 16 h. Volatiles were removed under reduced pressure, the residue was triturated with MeCN and washed with water to yield the title compound (13.2 g, 61.1 mmol, 24% yield).

UPLC-MS (254 nm): m/z (M+1) 217, rt=1.20 min.

### Intermediate 4: N-(5-(5-Chloro-2-fluorophenyl)-1H-imidazol-2-yl)acetamide

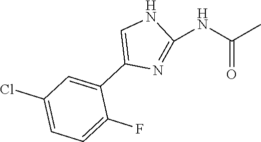 [SMILES: CC(=O)Nc1nc(-c2cc(Cl)ccc2F)c[nH]1]

Prepared from Intermediate 2 (1 g, 3.98 mmol) following similar procedure as for Intermediate 3. Purification by flash chromatography (gradient of elution from 0% to 100% of EtOAc in heptane) yielded the title compound (0.418 g, 1.648 mmol, 41% yield).

LC-MS (ESI): m/z (M+1) 254.0, rt=0.76 min (Method D).

### Intermediate 5: 1-(6-(6-Methylpyridin-2-yl)-2,3-dihydro-1H-imidazo[1,2-a]imidazol-1-yl)ethan-1-one

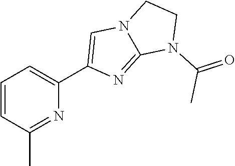 [SMILES: CC(=O)N1CCn2cc(-c3cccc(C)n3)nc21]

To a suspension of Intermediate 3 (13.2 g, 61.1 mmol) in MeCN (50 mL), K₂CO₃ (42.2 g, 305.4 mmol) was added followed by dropwise addition of a solution of 1,2-dibromoethane (16.0 mL, 183.3 mmol) in MeCN (30 mL). The reaction was heated to 80° C. and stirred for 16 h. Solvent was evaporated, the residue was dissolved in a DCM/MeOH mixture (4:1 v/v) and filtered through silica. Volatiles were removed under reduced pressure. The residue was crystallized from EtOAc to yield the title compound (7.5 g, 31.0 mmol, 51% yield) (major regioisomer).

UPLC-MS: m/z (M+1) 243.0, rt=1.20 min.

### Intermediate 6: 1-(5-Bromo-6-(6-methylpyridin-2-yl)-2,3-dihydro-1H-imidazo[1,2-a]imidazol-1-yl)ethan-1-one

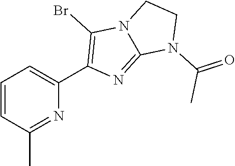 [SMILES: CC(=O)N1CCn2c1nc(-c1cccc(C)n1)c2Br]

NBS (7.4 g, 41.5 mmol) was added portionwise to a stirred solution of Intermediate 5 (10.0 g, 41.3 mmol) in dry DCM (15 mL), cooled at 0° C. The mixture was stirred for 30 min, then was diluted with water and extracted DCM. Combined organics were dried over Na₂SO₄, filtered and volatiles removed under reduced pressure to give the title compound (12.9 g, 40.3 mmol, 98% yield), which was used as such in the next step.

UPLC-MS: m/z (M+1) 320.9-322.9, rt=1.23 min.

### Intermediate 7: 1-(6-(5-Chloro-2-fluorophenyl)-2,3-dihydro-1H-imidazo[1,2-a]imidazol-1-yl)ethan-1-one

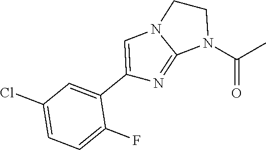 [SMILES: CC(=O)N1CCn2cc(-c3cc(Cl)ccc3F)nc21]

Prepared from Intermediate 4 (0.488 g, 1.924 mmol) following similar procedure as for Intermediate 5. Purification by RP flash chromatography (gradient of elution from 0% to 100% of B in A; A: water/MeCN 95:5+0.1% HCOOH, B: MeCN/water 95:5+0.1% HCOOH) yielded the title compound (0.187 g, 0.669 mmol, 35% yield) (major regioisomer).

LC-MS (ESI): m/z (M+1) 280.0, rt=0.73 min (Method D).

### Intermediate 8: 1-(5-Bromo-6-(5-chloro-2-fluorophenyl)-2,3-dihydro-1H-imidazo[1,2-a]imidazol-1-yl)ethan-1-one

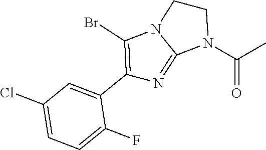 [SMILES: CC(=O)N1CCn2c1nc(-c1cc(Cl)ccc1F)c2Br]

Prepared from Intermediate 7 (0.187 g, 0.669 mmol) following the procedure used for the Intermediate 6. The crude title compound (assumed quantitative yield) was used as such in the next step.

LC-MS (ESI): m/z (M+1) 359.9-361.0, rt=1.09 min (Method D).

### Intermediate 9: N-(5-bromo-2-fluorophenyl)-3-(4-methylpiperazin-1-yl)propanamide

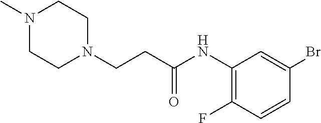 [SMILES: CN1CCN(CCC(=O)Nc2cc(Br)ccc2F)CC1]

A mixture of 5-bromo-2-fluoroaniline (0.400 g, 2.10 mmol), TEA (1.47 mL, 10.5 mmol), 3-bromopropanoyl chloride (0.170 mL, 1.68 mmol) in DCM (5.00 mL) was stirred at −78° C. for 2 h. The mixture was partitioned between DCM and water and the organic phase was filtered through a phase separator tube and concentrated under reduced pressure. The residue was taken up in THF and 1-methylpiperazine (0.23 mL, 2.10 mmol) was added. The resulting mixture was stirred at 60° C. for 1 h. The mixture was concentrated under reduced pressure and the crude was purified by flash chromatography (gradient of elution from 0 to 50% of B in A; A: DCM, B: DCM:MeOH 9:1) to give the title compound (0.562 g, 1.63 mmol, 78% yield).

LC-MS (ESI): m/z (M+1) 344.3, rt=0.40 min (Method D).

### Intermediate 10: 1-(5-(3-Amino-4-fluorophenyl)-6-(6-methylpyridin-2-yl)-2,3-dihydro-1H-imidazo[1,2-a]imidazol-1-yl)ethanone

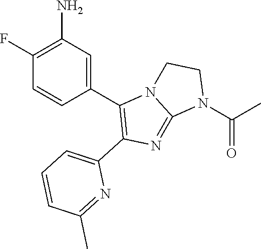 [SMILES: CC(=O)N1CCn2c1nc(-c1cccc(C)n1)c2-c1ccc(F)c(N)c1]

Prepared from Intermediate 5 (0.200 g, 0.825 mmol) and 5-bromo-2-fluoroaniline (0.118 g, 0.991 mmol), following procedure A, in the presence of Pd₂(dba)₃ (0.038 g, 0.041 mmol). Purification by RP flash chromatography (gradient of elution from 0 to 50% of B in A; A: water/MeCN 95:5+0.1% HCOOH, B: MeCN/water 95:5+0.1% HCOOH) yielded the title compound (0.243 g, 0.692 mmol, 84% yield).

LC-MS (ESI): m/z (M+1) 352.3, rt=0.42 min (Method D).

### Intermediate 11: N-(5-(1-acetyl-6-(6-methylpyridin-2-yl)-2,3-dihydro-1H-imidazo[1,2-a]imidazol-5-yl)-2-fluorophenyl)acrylamide

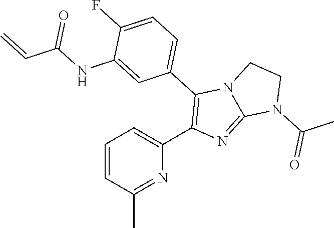 [SMILES: C=CC(=O)Nc1cc(-c2c(-c3cccc(C)n3)nc3n2CCN3C(C)=O)ccc1F]

To a cold (−45° C.) suspension of Intermediate 10 (0.150 g, 0.427 mmol) and TEA (0.30 mL, 2.136 mmol) in dry DCM (7.5 mL), 3-bromopropionyl chloride (0.095 g, 0.555 mmol) was added and the mixture was stirred at the same temperature for 30 min. The solution was diluted with DCM (5 mL) and washed with water (5 mL). The aqueous layer was extracted three times with DCM. Combined organics were washed with brine, dried over Na₂SO₄, filtered and dried under reduced pressure to give the crude product, which was used as such in the next step.

UPLC-MS: m/z (M+1) 406.9, rt=1.30 min.

### Intermediate 12: 2-Fluoro-4-(4,4,5,5-tetramethyl-1,3,2-dioxaborolan-2-yl)aniline

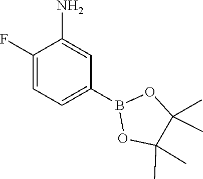 [SMILES: CC1(C)OB(c2ccc(F)c(N)c2)OC1(C)C]

To a suspension of 4-bromo-2-fluoroaniline (0.500 g, 2.63 mmol)) in dioxane (10 mL), bis(pinacolato)diboron (1.13 g, 4.47 mmol) and KOAc (1.09 g, 11.05 mmol) were added. The resulting mixture was saturated with argon before addition of Pd(dppf)Cl₂·DCM (0.20 g, 0.26 mmol). The mixture was sealed and stirred at 100° C. for 40 min. Suspension was diluted DCM, filtered through celite and volatiles removed under reduced pressure. Purification by flash chromatography (gradient of elution from 0 to 50% of EtOAc+5% TEA in hexane) yielded the title compound (0.581 g, 2.44 mmol, 93% yield).

UPLC-MS: m/z (M+1) 238.1, rt=1.67 min.

### Intermediate 13: 1-(5-(3-Amino-4-fluorophenyl)-6-(5-chloro-2-fluorophenyl)-2,3-dihydro-1H-imidazo[1,2-a]imidazol-1-yl)ethan-1-one

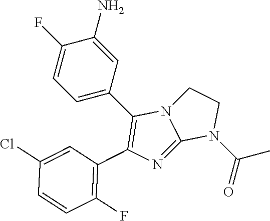 [SMILES: CC(=O)N1CCn2c1nc(-c1cc(Cl)ccc1F)c2-c1ccc(F)c(N)c1]

Prepared from Intermediate 8 (0.200 g, 0.56 mmol) and Intermediate 12 (0.400 g, 1.67 mmol) following general procedure B. Purification by flash chromatography (gradient of elution from 20 to 90% of EtOAc+5% TEA in hexane) yielded the title compound (0.112 g, mmml, 0.287 mmol, 42% yield).

UPLC-MS: m/z (M+1) 389.3, rt=1.60 min.

### Intermediate 14: N-(5-(1-acetyl-6-(6-methylpyridin-2-yl)-2,3-dihydro-1H-imidazo[1,2-a]imidazol-5-yl)-2-fluorophenyl)-3-(piperidin-1-yl)propanamide

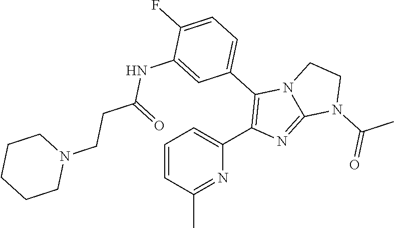 [SMILES: CC(=O)N1CCn2c1nc(-c1cccc(C)n1)c2-c1ccc(F)c(NC(=O)CCN2CCCCC2)c1]

To a suspension of Intermediate 11 (0.100 g, 0.247 mmol) in dry MeOH (4.2 mL), piperidine (37 μL, 0.370 mmol) and TEA (86 μL, 0.617 mmol) were added. The mixture was stirred at RT for 24 h, then volatiles were removed under reduced pressure. Purification by pTLC on silica gel using DCM:MeOH:TEA 9:0.5:0.5 (v/v/v) mixture as eluent, followed by trituration with MeOH yielded the title compound (0.035 g, 0.071 mmol, 29% yield).

UPLC-MS: m/z (M+1) 491.7, rt=1.23 min.

### Intermediate 15: N-(5-(1-acetyl-6-(6-methylpyridin-2-yl)-2,3-dihydro-1H-imidazo[1,2-a]imidazol-5-yl)-2-fluorophenyl)-3-(4-(2-hydroxyethyl)piperazin-1-yl)propanamide

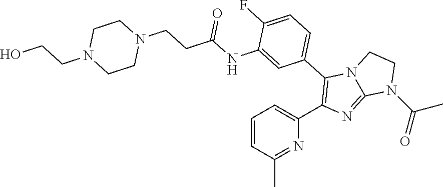 [SMILES: CC(=O)N1CCn2c1nc(-c1cccc(C)n1)c2-c1ccc(F)c(NC(=O)CCN2CCN(CCO)CC2)c1]

Prepared from Intermediate 11 (0.100 g, 0.247 mmol) according to the procedure described for Intermediate 14 and using 1-(2-hydroxyethyl)piperazine (46 μL, 0.370 mmol). Purification by pTLC on silica gel using DCM:MeOH:TEA 9:0.5:0.5 (v/v/v) mixture as eluent, followed by trituration with MeOH yielded the title compound (0.035 g, 0.065 mmol, 26% yield).

UPLC-MS: m/z (M+1) 536.7, rt=1.19 min.

### Intermediate 16: N-(5-(1-acetyl-6-(6-methylpyridin-2-yl)-2,3-dihydro-1H-imidazo[1,2-a]imidazol-5-yl)-2-fluorophenyl)-3-(4-(2,2,2-trifluoroethyl)piperazin-1-yl)propanamide

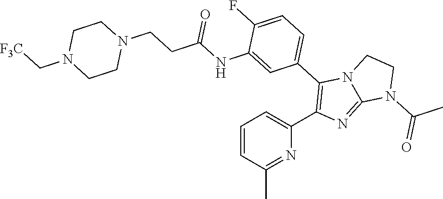 [SMILES: CC(=O)N1CCn2c1nc(-c1cccc(C)n1)c2-c1ccc(F)c(NC(=O)CCN2CCN(CC(F)(F)F)CC2)c1]

Prepared from Intermediate 11 (0.100 g, 0.247 mmol) according to the procedure described for Intermediate 14 and using 1-(2,2,2-trifluoroethyl)piperazine (87 μL, 0.192 mmol). Purification by pTLC on silica gel using DCM:MeOH:TEA 9:0.25:0.25 (v/v/v) mixture as eluent, followed by trituration with MeOH yielded the title compound (0.066 g, 0.115 mmol, 72% yield).

UPLC-MS: m/z (M+1) 574.7, rt=1.30 min.

### Intermediate 17: N-(5-(1-acetyl-6-(6-methylpyridin-2-yl)-2,3-dihydro-1H-imidazo[1,2-a]imidazol-5-yl)-2-fluorophenyl)-2-(dimethylamino)acetamide

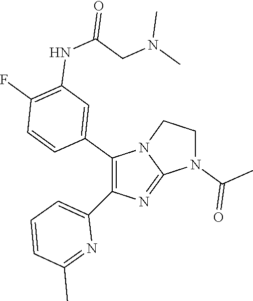 [SMILES: CC(=O)N1CCn2c1nc(-c1cccc(C)n1)c2-c1ccc(F)c(NC(=O)CN(C)C)c1]

A suspension of dimethylglycine (0.098 g, 0.95 mmol), HATU (0.432 g, 1.13 mmol) and DIPEA (0.330 mL, 1.90 mmol) in DMA (1.05 mL) was stirred at RT for 20 min. Then, Intermediate 10 (150 mg, 0.43 mmol) was added and the solution allowed to stir at RT for 18 h. Volatiles were removed under reduced pressure. Purification by flash chromatography on NH cartridge eluting with 2% MeOH in DCM yielded the title compound (0.120 g, 0.275 mmol, 64% yield).

UPLC-MS: m/z (M+1) 437.6, rt=1.20 min.

### Intermediate 18: N-(5-(1-acetyl-6-(6-methylpyridin-2-yl)-2,3-dihydro-1H-imidazo[1,2-a]imidazol-5-yl)-2-fluorophenyl)-2-(4-methylpiperazin-1-yl)acetamide

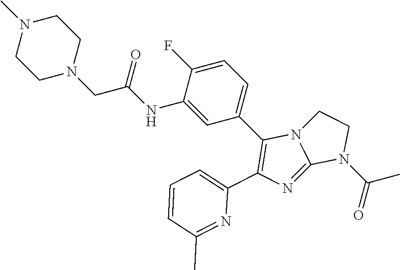 [SMILES: CC(=O)N1CCn2c1nc(-c1cccc(C)n1)c2-c1ccc(F)c(NC(=O)CN2CCN(C)CC2)c1]

Prepared from Intermediate 10 (0.150 mg, 0.430 mmol) following procedure described for Intermediate 17 and using 4-methylpiperazineacetic acid (0.150 mg, 0.949 mmol). Purification by flash chromatography on NH cartridge eluting with 2% MeOH in DCM followed by trituration in MeOH yielded the title compound (0.130 mg, 0.262 mmol, 62% yield).

UPLC-MS: m/z (M+1) 492.6, rt=1.20 min.

### Intermediate 19: N-(5-(1-acetyl-6-(6-methylpyridin-2-yl)-2,3-dihydro-1H-imidazo[1,2-a]imidazol-5-yl)-2-fluorophenyl)-4-(4-methylpiperazin-1-yl)butanamide

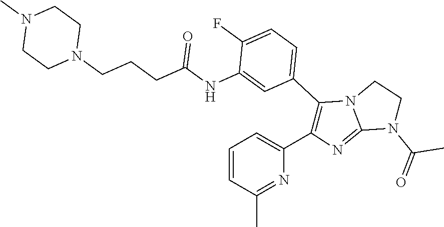 [SMILES: CC(=O)N1CCn2c1nc(-c1cccc(C)n1)c2-c1ccc(F)c(NC(=O)CCCN2CCN(C)CC2)c1]

Prepared from Intermediate 10 (0.05 g, 0.142 mmol) following procedure described for Intermediate 17 and using 4-(4-methylpiperazin-1-yl)butanoic acid (0.059 g, 0.320 mmol). Purification by flash chromatography on NH cartridge eluting with 2% MeOH in DCM followed by trituration in MeOH yielded the title compound (0.07 g, 0.135 mmol, 95% yield).

UPLC-MS: m/z (M+1) 520.8, rt=1.24 min.

### Intermediate 20: N-(5-(1-acetyl-6-(5-chloro-2-fluorophenyl)-2,3-dihydro-1H-imidazo[1,2-a]imidazol-5-yl)-2-fluorophenyl)-3-(4-methylpiperazin-1-yl)propanamide

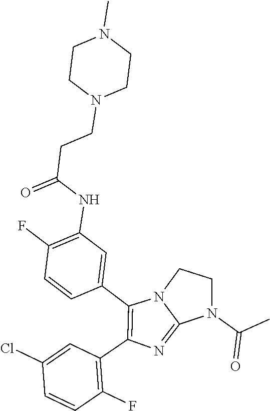 [SMILES: CC(=O)N1CCn2c1nc(-c1cc(Cl)ccc1F)c2-c1ccc(F)c(NC(=O)CCN2CCN(C)CC2)c1]

To a solution of Intermediate 13 (0.054 g, 0.139 mmol) in dry DCM (2.7 mL), TEA (0.1 mL, 0.70 mmol) was added and the mixture was cooled to −45° C. 3-Bromopropionyl chloride (18 μL, 0.18 mmol) was added and the mixture stirred for 30 min. Then, 1-methylpiperazine (30 μL, 0.46 mmol) was added dropwise and the solution was allowed to warm to RT and stirred for 1 h. The reaction mixture was quenched with water and diluted with DCM. The two phases were separated and the aqueous layer was extracted with DCM. Combined organics were dried over Na₂SO₄, filtered and volatiles removed under reduced pressure. Purification by pTLC on silica gel eluting with 70% EtOAc+5% TEA in hexane yielded the title compound (0.037 g, 0.068 mmol, 49% yield).

UPLC-MS: m/z (M+1) 543.6, rt=1.45 min.

### Intermediate 21: N-(5-(1-acetyl-6-(6-methylpyridin-2-yl)-2,3-dihydro-1H-imidazo[1,2-a]imidazol-5-yl)-2-fluorophenyl)-3-(dimethylamino)propanamide

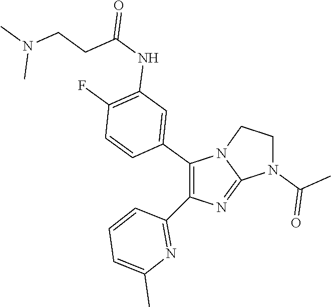 [SMILES: CC(=O)N1CCn2c1nc(-c1cccc(C)n1)c2-c1ccc(F)c(NC(=O)CCN(C)C)c1]

Freshly-prepared Intermediate 11 was synthesized from Intermediate 10 (0.050 g, 0.142 mmol) as described before, and treated with 2M solution of dimethylamine in THF (0.2 mL, 0.400 mmol) added dropwise at −45° C. The reaction mixture was allowed to warm to RT and stirred for 2 h. The mixture was diluted with DCM and quenched with water. The two phases were separated and the organic layer was dried over Na₂SO₄, filtered and volatiles removed under reduced pressure to yield the crude title compound (0.05 g), which was used as such in the next step.

UPLC-MS: m/z (M+1) 451.6, rt=1.19 min.

### Intermediate 22: N-(5-(1-acetyl-6-(5-chloro-2-fluorophenyl)-2,3-dihydro-1H-imidazo[1,2-a]imidazol-5-yl)-2-fluorophenyl)-3-(piperidin-1-yl)propanamide

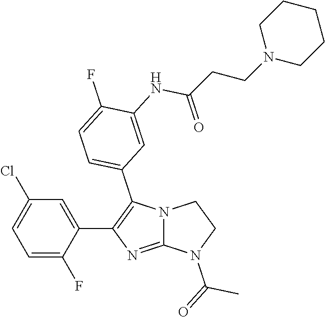 [SMILES: CC(=O)N1CCn2c1nc(-c1cc(Cl)ccc1F)c2-c1ccc(F)c(NC(=O)CCN2CCCCC2)c1]

Prepared from Intermediate 13 (0.112 g, 0.289 mmol) following the procedure used for Intermediate 20 and using piperidine (0.073 g, 0.867 mmol). Volatiles were removed under reduced pressure to yield the crude title compound (0.109 g), which was used as such in the next step.

UPLC-MS: m/z (M+1) 528.6, rt=1.46 min.

### Intermediate 23: N-(5-(1-acetyl-6-(6-methylpyridin-2-yl)-2,3-dihydro-1H-imidazo[1,2-a]imidazol-5-yl)-2-fluorophenyl)-4-(piperidin-1-yl)butanamide

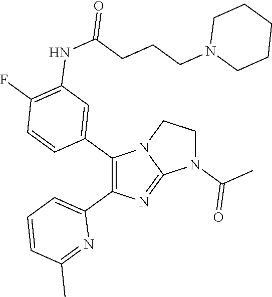 [SMILES: CC(=O)N1CCn2c1nc(-c1cccc(C)n1)c2-c1ccc(F)c(NC(=O)CCCN2CCCCC2)c1]

To a stirred mixture of Intermediate 10 (0.075 g, 0.21 mmol), 4-(piperidin-1-yl)butanoic acid hydrochloride (0.06 g, 0.28 mmol), and DMAP (6 mg, 0.05 mmol) in DCM (3.5 mL), EDC·HCl (0.123 g, 0.64 mmol) was added and the resulting reaction mixture was stirred at RT for 4 h. The mixture was diluted with DCM and washed with a sat. aq. NaHCO₃ solution. The organic phase was dried over Na₂SO₄, and the solvent removed under reduced pressure. Purification by flash chromatography (NH cartridge, gradient of elution from 0 to 30% of B in A; A: DCM, B: DCM:MeOH 9:1) yielded the title compound (0.060 g, 0.12 mmol, 56% yield).

LC-MS (ESI): m/z (M-I+1) 505.3, rt=0.90 min (Method E).

### Intermediate 24: 1-(4-((5-(1-Acetyl-6-(6-methylpyridin-2-yl)-2,3-dihydro-1H-imidazo[1,2-a]imidazol-5-yl)-2-fluorophenyl)amino)-4-oxobutyl)-1-methylpiperidin-1-ium iodide

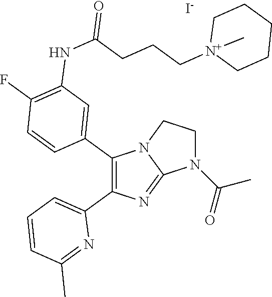

To a stirred solution of Intermediate 23 (0.095 g, 0.19 mmol) in MeOH (0.40 mL) and acetone (5.8 mL), at RT, iodomethane (0.06 mL, 0.96 mmol) was added and the resulting reaction mixture was stirred at RT for 12 h. The mixture was filtered, the solid was washed with acetone and dried under vacuum to yield the crude title compound (0.11 g), which was used as such in the next step.

LC-MS (ESI): m/z (M-I+1) 519.3, rt=0.95 min (Method E).

### Intermediate 25: 1-(5-(4-fluoro-3-((1-isopropylpiperidin-4-yl)amino)phenyl)-6-(6-methylpyridin-2-yl)-2,3-dihydro-1H-imidazo[1,2-a]imidazol-1-yl)ethan-1-one

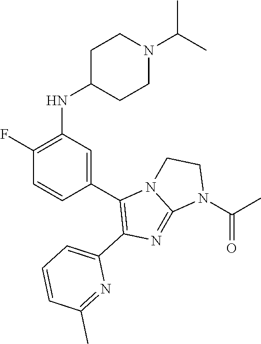 [SMILES: CC(=O)N1CCn2c1nc(-c1cccc(C)n1)c2-c1ccc(F)c(NC2CCN(C(C)C)CC2)c1]

To a solution of Intermediate 10 (0.090 g, 0.256 mmol) in DCM (2 mL), 1-methylpiperidine-4-carbaldehyde (0.036 g, 0.282 mmol), acetic acid (drops) and STAB (0.0059 g, 0.282 mmol) were added and the reaction stirred for 1 h. Excess of STAB, AcOH and 1-methylpiperidine-4-carbaldehyde was added overtime allowing the reaction to stir at RT for additional 7 h. Reaction was quenched with sat. aq. NaHCO₃ solution. The two phases were separated, the organic phase was filtered through a phase separator tube and volatiles were removed under reduced pressure. Purification by flash chromatography (gradient of elution of from 0 to 100% of B in A; A: DCM, B: DCM:MeoH 9:1) yielded the title compound (40 mg, 0.086 mmol, 34% yield).

LC-MS (ESI): m/z (M+1) 477.4, rt=0.34 min (Method D).

### Intermediate 26: 1-(5-(4-fluoro-3-(((1-methylpiperidin-4-yl)methyl)amino)phenyl)-6-(6-methylpyridin-2-yl)-2,3-dihydro-1H-imidazo[1,2-a]imidazol-1-yl)ethan-1-one

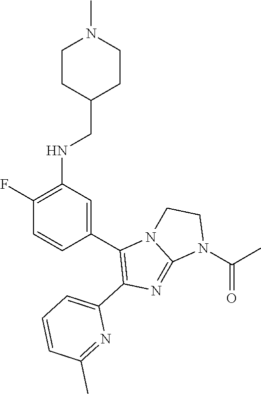 [SMILES: CC(=O)N1CCn2c1nc(-c1cccc(C)n1)c2-c1ccc(F)c(NCC2CCN(C)CC2)c1]

To a solution of Intermediate 10 (0.050 g, 0.142 mmol) in DCM (1.5 mL), 1-isopropylpiperidin-4-one (0.021 mL, 0.142 mmol), acetic acid (9.78 μl, 0.171 mmol) and STAB (45 mg, 0.213 mmol) were added and the reaction stirred for 1 h. Excess of STAB, AcOH and 1-isopropylpiperidin-4-one was added overtime allowing the reaction to stir at RT for 7 days. Reaction was diluted with DCM and quenched with sat. aq. NaHCO₃ solution. The two phases were separated and the aqueous layer was extracted with DCM. Combined organics were filtered through a phase separator tube and volatiles removed under reduced pressure. Purification by RP flash chromatography (gradient of elution of from 0 to 25% of B in A; A: water/MeCN 95:5+0.1% HCOOH, B: MeCN/water 95:5+0.1% HCOOH) yielded the title compound (23 mg, 0.048 mmol, 34% yield).

LC-MS (ESI): m/z (M+1) 463.4, rt=0.32 min (Method D).

### PREPARATION OF THE EXAMPLES

### Example 1: N-(2-fluoro-5-(6-(6-methylpyridin-2-yl)-2,3-dihydro-1H-imidazo[1,2-a]imidazol-5-yl)phenyl)-3-(4-methylpiperazin-1-yl)propanamide

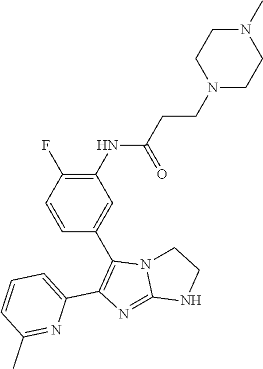 [SMILES: Cc1cccc(-c2nc3n(c2-c2ccc(F)c(NC(=O)CCN4CCN(C)CC4)c2)CCN3)n1]

Prepared from Intermediate 5 (0.080 g, 0.330 mmol) and Intermediate 9 (0.136 g, 0.396 mmol), following procedure A, in the presence of Pd₂(dba)₃ (15 mg, 0.017 mmol) at 160° C. Purification by flash chromatography (gradient of elution from 0 to 100% of B in A; A: DCM, B: DCM:EtOH 9:1) yielded the title compound (32.4 mg, 0.07 mmol, 21% yield).

LC-MS (ESI): m/z (M+1) 464.4, rt=0.28 min (Method D).

¹H NMR (400 MHz, DMSO-d6) δ ppm 10.48 (s, 1H) 8.32 (br d, J=6.36 Hz, 1H) 7.54-7.62 (m, 2H) 7.30-7.37 (m, 1H) 7.21-7.30 (m, 1H) 6.95-7.02 (m, 1H) 4.24-4.38 (m, 2H) 4.02-4.17 (m, 2H) 2.52-2.61 (m, 8H) 2.30-2.39 (m, 4H) 2.23 (s, 3H) 2.13 (s, 3H).

### Example 2: N-(2-fluoro-5-(6-(6-methylpyridin-2-yl)-2,3-dihydro-1H-imidazo[1,2-a]imidazol-5-yl)phenyl)-3-(piperidin-1-yl)propanamide

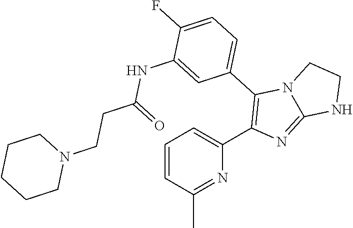 [SMILES: Cc1cccc(-c2nc3n(c2-c2ccc(F)c(NC(=O)CCN4CCCCC4)c2)CCN3)n1]

Prepared according to procedure D, starting from Intermediate 14 (0.035 g, 0.071 mmol). Volatiles were removed under reduced pressure to yield the title compound (0.03 g, 0.067 mmol, 93% yield).

LC-MS (ESI): m/z (M+1) 449.04, rt=2.55 min (Method A).

¹H NMR (400 MHz, DMSO-d₆) δ 10.74 (s, 1H), 8.36 (dd, J=7.7, 2.3 Hz, 1H), 7.62-7.47 (m, 2H), 7.37 (ddd, J=8.7, 4.9, 2.3 Hz, 1H), 7.22 (dd, J=10.9, 8.6 Hz, 1H), 6.95 (d, J=7.1 Hz, 1H), 6.23-6.12 (m, 1H), 4.01 (dd, J=9.0, 6.7 Hz, 2H), 3.96-3.78 (m, 2H), 2.59 (t, J=6.3 Hz, 2H), 2.43 (s, 4H), 2.25 (s, 3H), 1.55 (t, J=5.7 Hz, 4H), 1.42 (s, 2H).

### Example 3: N-(2-fluoro-5-(6-(6-methylpyridin-2-yl)-2,3-dihydro-1H-imidazo[1,2-a]imidazol-5-yl)phenyl)-3-(4-(2-hydroxyethyl)piperazin-1-yl)propanamide

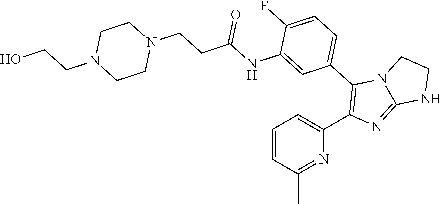 [SMILES: Cc1cccc(-c2nc3n(c2-c2ccc(F)c(NC(=O)CCN4CCN(CCO)CC4)c2)CCN3)n1]

Prepared according to procedure D, starting from Intermediate 15 (0.035 g, 0.065 mmol). Volatiles were removed under reduced pressure to yield the title compound (0.03 g, 0.061 mmol, 93% yield).

LC-MS (ESI): m/z (M+1) 494.16, rt=2.43 min (Method A).

¹H NMR (400 MHz, DMSO-d₆) δ 10.49 (s, 1H), 8.33 (dd, J=7.9, 2.2 Hz, 1H), 7.61-7.48 (m, 2H), 7.38 (ddd, J=7.4, 4.8, 2.2 Hz, 1H), 7.23 (dd, J=10.8, 8.6 Hz, 1H), 6.95 (dd, J=7.4, 1.2 Hz, 1H), 6.22-6.16 (m, 1H), 4.37 (t, J=5.4 Hz, 1H), 4.01 (dd, J=8.9, 6.7 Hz, 2H), 3.93-3.80 (m, 2H), 3.49 (q, J=6.0 Hz, 2H), 2.61 (t, J=6.5 Hz, 2H), 2.52-2.40 (buried m, 7H), 2.38 (t, J=6.3 Hz, 2H).

### Example 4: N-(2-fluoro-5-(6-(6-methylpyridin-2-yl)-2,3-dihydro-1H-imidazo[1,2-a]imidazol-5-yl)phenyl)-3-(4-(2,2,2-trifluoroethyl)piperazin-1-yl)propanamide

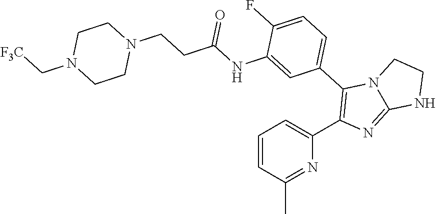 [SMILES: Cc1cccc(-c2nc3n(c2-c2ccc(F)c(NC(=O)CCN4CCN(CC(F)(F)F)CC4)c2)CCN3)n1]

Prepared according to procedure D, starting from Intermediate 16 (0.033 g, 0.052 mmol). Purification by pTLC on NH-silica plate eluting with 4% MeOH in DCM yielded the title compound (0.023 g, 0.043 mmol, 83% yield).

LC-MS (ESI): m/z (M+1) 532.14, rt=2.74 min (Method A).

¹H NMR (400 MHz, DMSO-d₆) δ 10.37 (s, 1H), 8.30 (dd, J=7.7, 2.3 Hz, 1H), 7.60-7.47 (m, 2H), 7.38 (ddd, J=7.5, 4.9, 2.3 Hz, 1H), 7.22 (dd, J=10.8, 8.6 Hz, 1H), 6.94 (d, J=7.2 Hz, 1H), 6.21-6.15 (m, 1H), 4.01 (dd, J=8.9, 6.7 Hz, 2H), 3.84 (t, J=8.2 Hz, 2H), 3.20-3.10 (m, 2H), 2.63 (dd, J=13.0, 5.7 Hz, 6H), 2.53-2.49 (buried m, 8H), 2.24 (s, 3H).

### Example 5: 2-(Dimethylamino)-N-(2-fluoro-5-(6-(6-methylpyridin-2-yl)-2,3-dihydro-1H-imidazo[1,2-a]imidazol-5-yl)phenyl)acetamide

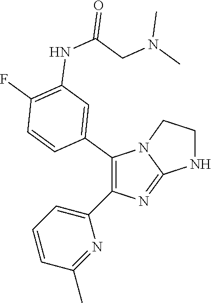 [SMILES: Cc1cccc(-c2nc3n(c2-c2ccc(F)c(NC(=O)CN(C)C)c2)CCN3)n1]

Prepared according to procedure D, starting from Intermediate 17 (0.033 g, 0.075 mmol). Purification by pTLC on NH-silica plate eluting with 4% MeOH in DCM yielded the title compound (0.023 g, 0.058 mmol, 77% yield).

LC-MS (ESI): m/z (M+1) 395.09, rt=3.00 min (Method C).

¹H NMR (400 MHz, DMSO-d₆) δ 9.51 (s, 1H), 8.22 (dd, J=7.7, 2.2 Hz, 1H), 7.61-7.48 (m, 2H), 7.42 (ddd, J=8.7, 4.9, 2.3 Hz, 1H), 7.23 (dd, J=10.7, 8.6 Hz, 1H), 6.94 (dd, J=7.5, 1.2 Hz, 1H), 6.22-6.16 (m, 1H), 4.02 (dd, J=8.9, 6.7 Hz, 2H), 3.89-3.80 (m, 2H), 3.10 (s, 2H), 2.30 (s, 6H), 2.24 (s, 3H).

### Example 6: N-(2-fluoro-5-(6-(6-methylpyridin-2-yl)-2,3-dihydro-1H-imidazo[1,2-a]imidazol-5-yl)phenyl)-2-(4-methylpiperazin-1-yl)acetamide

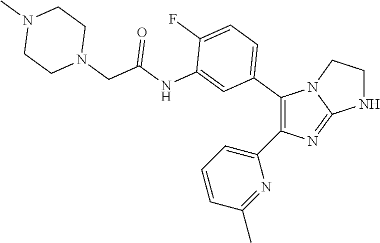 [SMILES: Cc1cccc(-c2nc3n(c2-c2ccc(F)c(NC(=O)CN4CCN(C)CC4)c2)CCN3)n1]

Prepared according to procedure D, starting from Intermediate 18 (0.033 g, 0.067 mmol).

Purification by pTLC on NH-silica plate eluting with 4% MeOH in DCM yielded the title compound (0.024 g, 0.053 mmol, 79% yield).

LC-MS (ESI): m/z (M+1) 449.89, rt=3.32 min (Method C).

¹H NMR (400 MHz, DMSO-d₆) δ 9.60-9.56 (m, 1H), 8.30 (dd, J=7.7, 2.3 Hz, 1H), 7.60-7.50 (m, 2H), 7.42 (ddd, J=8.5, 5.0, 2.3 Hz, 1H), 7.25 (dd, J=10.7, 8.6 Hz, 1H), 6.95 (dd, J=7.4, 1.2 Hz, 1H), 6.22-6.18 (m, 1H), 4.02 (dd, J=8.9, 6.6 Hz, 2H), 3.86 (td, J=8.5, 7.1, 4.2 Hz, 2H), 3.15 (s, 2H), 2.54-2.51 (buried m, 4H), 2.38 (br s, 4H), 2.24 (s, 3H), 2.18 (s, 3H).

### Example 7: N-(2-fluoro-5-(6-(6-methylpyridin-2-yl)-2,3-dihydro-1H-imidazo[1,2-a]imidazol-5-yl)phenyl)-4-(4-methylpiperazin-1-yl)butanamide

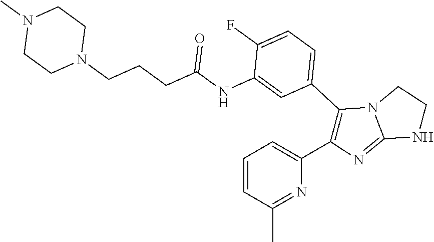 [SMILES: Cc1cccc(-c2nc3n(c2-c2ccc(F)c(NC(=O)CCCN4CCN(C)CC4)c2)CCN3)n1]

Prepared according to procedure D, starting from Intermediate 19 (0.034 g, 0.065 mmol).

Purification by pTLC on NH-silica plate eluting with 4% MeOH in DCM yielded the title compound (0.024 g, 0.050 mmol, 77% yield).

LC-MS (ESI): m/z (M-1) 476.00, rt=2.38 min (Method A).

¹H NMR (400 MHz, DMSO-d₆) δ 9.65 (s, 1H), 8.14 (d, J=7.4 Hz, 1H), 7.59-7.49 (m, 2H), 7.39 (d, J=6.4 Hz, 1H), 7.20 (dd, J=10.7, 8.6 Hz, 1H), 6.97-6.92 (m, 1H), 6.19 (s, 1H), 4.02 (t, J=7.8 Hz, 2H), 3.85 (t, J=7.8 Hz, 2H), 2.54-2.51 (buried in, 8H), 2.37 (d, J=7.4 Hz, 4H), 2.28 (t, J=7.1 Hz, 5H), 2.25 (s, 3H), 2.12 (s, 3H), 1.72 (p, J=7.3 Hz, 2H).

### Example 8: N-(5-(6-(5-chloro-2-fluorophenyl)-2,3-dihydro-1H-imidazo[1,2-a]imidazol-5-yl)-2-fluorophenyl)-3-(4-methylpiperazin-1-yl)propanamide

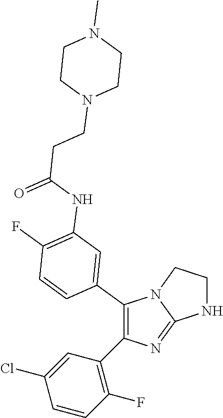 [SMILES: CN1CCN(CCC(=O)Nc2cc(-c3c(-c4cc(Cl)ccc4F)nc4n3CCN4)ccc2F)CC1]

Prepared according to procedure D, starting from Intermediate 20 (0.037 g, 0.068 mmol).

Purification by pTLC on NH-silica plate eluting with 4% MeOH in DCM yielded the title compound (0.019 g, 0.038 mmol, 56% yield).

LC-MS (ESI): m/z (M+1) 501.05, rt=1.54 min (Method B).

¹H NMR (400 MHz, DMSO-d₆) δ 10.62 (s, 1H), 8.14-8.04 (m, 1H), 7.51 (dd, J=6.3, 2.8 Hz, 1H), 7.31 (ddd, J=8.7, 4.2, 2.8 Hz, 1H), 7.22 (dd, J=10.9, 8.5 Hz, 1H), 7.12 (dd, J=9.9, 8.8 Hz, 1H), 6.95-6.85 (m, 1H), 6.24 (d, J=2.7 Hz, 1H), 4.07 (dd, J=9.0, 6.8 Hz, 2H), 3.93-3.79 (m, 2H), 2.59 (t, J=6.4 Hz, 4H), 2.54-2.51 (buried m, 8H), 2.16 (s, 3H).

### Example 9: 3-(Dimethylamino)-N-(2-fluoro-5-(6-(6-methylpyridin-2-yl)-2,3-dihydro-1H-imidazo[1,2-a]imidazol-5-yl)phenyl)propanamide

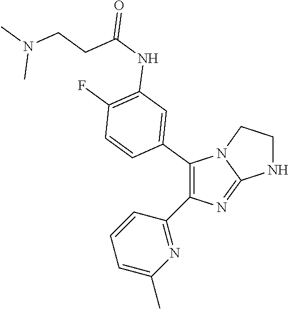 [SMILES: Cc1cccc(-c2nc3n(c2-c2ccc(F)c(NC(=O)CCN(C)C)c2)CCN3)n1]

Prepared according to procedure D, starting from Intermediate 21 (0.050 g, 0.111 mmol).

Purification by pTLC on NH-silica plate eluting with 4% MeOH in DCM yielded the title compound (0.014 g, 0.034 mmol, 31% yield).

LC-MS (ESI): m/z (M+1) 409.17, rt=2.43 min (Method A).

¹H NMR (400 MHz, DMSO-d₆) δ 10.36 (s, 1H), 8.27 (dd, J=7.9, 2.3 Hz, 1H), 7.62-7.43 (m, 2H), 7.37 (ddd, J=8.5, 4.8, 2.3 Hz, 1H), 7.20 (dd, J=10.8, 8.6 Hz, 1H), 6.99-6.90 (m, 1H), 6.18 (t, J=2.6 Hz, 1H), 4.01 (dd, J=8.9, 6.6 Hz, 2H), 3.93-3.77 (m, 2H), 2.54-2.50 (m, 4H), 2.24 (s, 3H), 2.20 (s, 6H).

### Example 10: N-(5-(6-(5-chloro-2-fluorophenyl)-2,3-dihydro-1H-imidazo[1,2-a]imidazol-5-yl)-2-fluorophenyl)-3-(piperidin-1-yl)propanamide

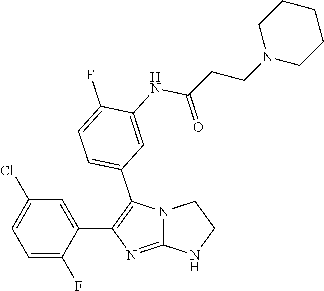 [SMILES: O=C(CCN1CCCCC1)Nc1cc(-c2c(-c3cc(Cl)ccc3F)nc3n2CCN3)ccc1F]

Prepared according to procedure D, starting from Intermediate 22 (0.109 g, 0.206 mmol).

Purification by pTLC on NH-silica plate eluting with 4% MeOH in DCM, followed by trituration with MeCN yielded the title compound (0.024 g, 0.049 mmol, 24% yield).

LC-MS (ESI): m/z (M+1) 486.06, rt=1.60 min (Method B).

¹H NMR (400 MHz, DMSO-d₆) δ 10.89 (s, 1H), 8.11 (dd, J=7.6, 2.3 Hz, 1H), 7.51 (dd, J=6.3, 2.8 Hz, 1H), 7.31 (ddd, J=8.7, 4.2, 2.8 Hz, 1H), 7.21 (dd, J=11.0, 8.6 Hz, 1H), 7.12 (dd, J=9.9, 8.8 Hz, 1H), 6.93-6.84 (m, 1H), 6.28-6.20 (m, 1H), 4.07 (dd, J=9.0, 6.7 Hz, 2H), 3.91-3.81 (m, 2H), 2.56 (m, 2H), 2.41 (m, 2H), 1.54 (t, J=5.7 Hz, 4H), 1.41 (s, 2H).

### Example 11: N-(2-fluoro-5-(6-(6-methylpyridin-2-yl)-2,3-dihydro-1H-imidazo[1,2-a]imidazol-5-yl)phenyl)-4-(piperidin-1-yl)butanamide

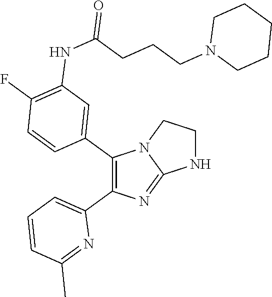 [SMILES: Cc1cccc(-c2nc3n(c2-c2ccc(F)c(NC(=O)CCCN4CCCCC4)c2)CCN3)n1]

Prepared according to procedure C, starting from Intermediate 23 (0.062 g, 0.12 mmol). Purification by RP flash chromatography (gradient of elution from 0 to 80% of B in A; A: water+0.2% NH₄OH, B: MeCN) yielded the title compound (0.010 g, 0.023 mmol, 18% yield).

LC-MS (ESI): m/z (M+1) 463.3, rt=0.40 min (Method D).

¹H NMR (400 MHz, CDCl₃) δ ppm 8.75 (br s, 1H), 8.36 (dd, J=7.3, 1.8 Hz, 1H), 7.39-7.46 (m, 1H), 7.34-7.38 (m, 1H), 7.31 (ddd, J=8.6, 5.1, 2.1 Hz, 1H), 6.99 (dd, J=10.7, 8.8 Hz, 1H), 6.89 (d, J=7.3 Hz, 1H), 4.12-4.19 (m, 2H), 4.04-4.11 (m, 1H), 3.95-4.03 (m, 2H), 2.47 (t, J=6.8 Hz, 2H), 2.30-2.43 (m, 9H), 1.89 (quin, J=6.6 Hz, 2H), 1.51-1.74 (m, 4H), 1.36-1.49 (m, 2H).

### Example 12: 1-(4-((2-Fluoro-5-(6-(6-methylpyridin-2-yl)-2,3-dihydro-1H-imidazo[1,2-a]imidazol-5-yl)phenyl)amino)-4-oxobutyl)-1-methylpiperidin-1-ium chloride hydrochloride

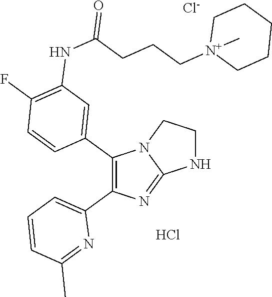

Prepared according to procedure D, starting from Intermediate 24 (0.05 g, 0.08 mmol). Purification by RP flash chromatography (gradient of elution from 0 to 75% of B in A; A: water+0.1% HCOOH, B: MeCN+0.1% HCOOH) yielded the compound as bis formate salt (0.032 g). This material was dissolved in MeOH (4 mL) and Dowex® Marathon™ A2 Chloride Form (0.08 mmol) was added. The mixture was shaken for 30 min at RT, then filtered and the resin was washed with MeOH. Filtrate was concentrated under reduced pressure to yield the title compound (0.012 g, 0.025 mmol, 33% yield).

LC-MS (ESI): m/z (M-2Cl+1) 477.3, rt=0.38 min (Method D).

¹H NMR (400 MHz, DMSO-d₆) δ ppm 9.98 (s, 1H), 8.00-8.29 (m, 1H), 7.54-7.73 (m, 1H), 7.36-7.48 (m, 1H), 7.24-7.34 (m, 1H), 7.04 (br d, J=7.6 Hz, 1H), 7.00 (br s, 2H), 4.03-4.16 (m, 2H), 3.87-3.99 (m, 2H), 3.26-3.41 (m, 6H), 3.01 (s, 3H), 2.51-2.57 (m, 2H), 2.34 (s, 3H), 1.87-2.04 (m, 2H), 1.78 (br s, 4H), 1.43-1.65 (m, 2H).

### Example 13: N-(2-fluoro-5-(6-(6-methylpyridin-2-yl)-2,3-dihydro-1H-imidazo[1,2-a]imidazol-5-yl)phenyl)-1-isopropylpiperidin-4-amine

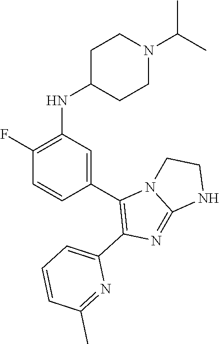 [SMILES: Cc1cccc(-c2nc3n(c2-c2ccc(F)c(NC4CCN(C(C)C)CC4)c2)CCN3)n1]

Prepared according to procedure C, starting from Intermediate 25 (0.023 g, 0.048 mmol). Purification by SCX cartridge (2 g, elution with 2N NH₃ in MeOH) yielded the title compound (0.017 g, 0.039 mmol, 81% yield).

LC-MS (ESI): m/z (M+1) 435.4, rt=0.35 min (Method D).

¹H NMR (400 MHz, DMSO-d₆) δ ppm 7.51 (t, J=7.72 Hz, 1H), 7.37 (d, J=7.94 Hz, 1H), 6.87-6.98 (m, 2H), 6.81 (br d, J=7.50 Hz, 1H), 6.62-6.74 (m, 1H), 6.04 (br s, 1H), 4.97 (br s, 1H), 3.91-4.02 (m, 2H), 3.74-3.83 (m, 2H), 3.02 (br s, 1H), 2.68 (br s, 2H), 2.25 (s, 3H), 2.06 (br s, 2H), 1.75 (br s, 2H), 1.37 (br s, 2H), 0.92 (br s, 6H).

### Example 14: 2-Fluoro-N-((1-methylpiperidin-4-yl)methyl)-5-(6-(6-methylpyridin-2-yl)-2,3-dihydro-1H-imidazo[1,2-a]imidazol-5-yl)aniline

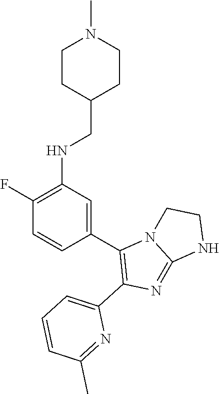 [SMILES: Cc1cccc(-c2nc3n(c2-c2ccc(F)c(NCC4CCN(C)CC4)c2)CCN3)n1]

Prepared according to procedure C, starting from Intermediate 26 (0.040 g, 0.086 mmol). Purification by RP flash chromatography (gradient of elution of from 0 to 30% of B in A; A: water/MeCN 95:5+0.1% HCOOH, B: MeCN/water 95:5+0.1% HCOOH) yielded the title compound (0.014 g, 0.033 mmol, 38% yield).

LC-MS (ESI): m/z (M+1) 421.5, rt=0.35 min (Method D).

¹H NMR (400 MHz, DMSO-d₆) δ ppm 7.55 (t, J=7.67 Hz, 1H) 7.42 (d, J=7.89 Hz, 1H) 6.92-7.01 (m, 2H) 6.83 (br d, J=8.55 Hz, 1H) 6.69-6.74 (m, 1H) 6.08 (br s, 1H) 5.34-5.40 (m, 1H) 4.01 (br t, J=7.67 Hz, 2H) 3.83 (br t, J=7.02 Hz, 2H) 2.88 (br t, J=6.14 Hz, 2H) 2.72 (br d, J=11.40 Hz, 2H) 2.29 (s, 3H) 2.14 (s, 3H) 1.79 (br t, J=11.29 Hz, 2H) 1.63 (br d, J=12.06 Hz, 2H) 1.34-1.52 (m, 1H) 1.05-1.18 (m, 2H).

### Pharmacological Activity of the Compounds of the Invention

### In Vitro Assay

The enzymatic activity of compounds of the present invention was monitored measuring the formation of ADP using the ADP-GLO Kinases assay. Following the incubation of the purified enzyme, a substrate and ATP, the produced ADP was converted into ATP, which in turn was converted into light by Ultra-Glo Luciferase. The luminescent signal positively correlated with ADP amount and kinase activity. Briefly, the kinase reaction was performed by incubating 2.6 nM of the purified, commercially available human ALK5 (recombinant TGF β1 N-term GST-tagged, 80-end), a final concentration of TGFβ1 peptide 94.5 μM (Promega, T36-58) and ultra-pure ATP (Promega V915B). The ATP concentration was set at the Km value (concentration of substrate which permits the enzyme to achieve half maximal velocity (Vmax)) of ALK5 (0.5 μM). Compound and ALK5 kinase were mixed and incubated for 15 min. Reactions were initiated by addition of ATP at a final concentration in the assay of 0.83 μM. After an incubation of 120 min, the reaction was stopped, and ADP production detected with ADP-Glo kit according to manufacturer's indications. All reaction and incubation steps were performed at 25° C. and the assays were performed in 384-well format and validated using a selection of reference compounds tested in 11-point concentration-response curve.

The results for individual compounds are provided below in Table 3 wherein the compounds are classified in term of potency with respect to their inhibitory activity on ALK5 receptor. Results were expressed as pIC₅₀ (negative logarithm of IC₅₀) and subsequently converted to pKᵢ (negative logarithm of dissociate function Kᵢ) using the Cheng-Prusoff equation. The higher the value of pKᵢ, the greater the inhibition of ALK5 activity.

TABLE 3

| Example No | ALK5 pKᵢ |
|---|---|
| 2, 9, 10 | ≥9.0 |
| 1, 3, 4, 7, 8, 11, 12, 14 | 8.1-8.9 |
| 5, 6, 13 | 7.5-8.0 |

As it can be appreciated, compounds of Table 3 show a good activity as antagonists of ALK5 receptor.

1: A compound of formula (I) 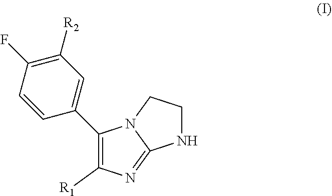
wherein
R₁ is selected from the group consisting of pyridyl optionally substituted by one or more —(C₁-C₆)alkyl, and phenyl optionally substituted by one or more halogen atoms;
R₂ is selected from the group consisting of —NH-heterocycloalkyl, wherein said heterocycloalkyl is optionally substituted by one or more —(C₁-C₆)alkyl; —NH—(C₁-C₆)alkylene-heterocycloalkyl, wherein said heterocycloalkyl is optionally substituted by one or more —(C₁-C₆)alkyl; —NH—C(O)—(C₁-C₆)alkylene-NRaRb; and —NH—C(O)—(C₁-C₆)alkylene-heterocycloalkyl, wherein said heterocycloalkyl is optionally substituted by one or more groups selected from the group consisting of —(C₁-C₆)alkyl, —(C₁-C₆)haloalkyl and —(C₁-C₆)hydroxyalkyl;
Ra is —(C₁-C₆)alkyl;
Rb is —(C₁-C₆)alkyl;
or a pharmaceutically acceptable salt thereof.

2: The compound of formula (I) or salt thereof according to claim 1, wherein R₁ is selected from the group consisting of 6-methylpyridin-2-yl and -(5-chloro-2-fluorophenyl); and R₂ is selected from the group consisting of -3-(4-methylpiperazin-1-yl)propanamide, -3-(piperidin-1-yl)propanamide, -3-(4-(2-hydroxyethyl)piperazin-1-yl)propanamide, -3-(4-(2,2,2-trifluoroethyl)piperazin-1-yl)propanamide, 2-(dimethylamino)acetamide, -2-(4-methylpiperazin-1-yl)acetamide, -4-(4-methylpiperazin-1-yl)butanamide, 3-(dimethylamino)propanamide, -4-(piperidin-1-yl)butanamide and 1-(4-amino-4-oxobutyl)-1-methylpiperidin-1-ium.

3: The compound of formula (I) or salt thereof according to claim 1, selected from the group consisting of:
N-(2-fluoro-5-(6-(6-methylpyridin-2-yl)-2,3-dihydro-1H-imidazo[1,2-a]imidazol-5-yl)phenyl)-3-(4-methylpiperazin-1-yl)propanamide;
N-(2-fluoro-5-(6-(6-methylpyridin-2-yl)-2,3-dihydro-1H-imidazo[1,2-a]imidazol-5-yl)phenyl)-3-(piperidin-1-yl)propanamide;
N-(2-fluoro-5-(6-(6-methylpyridin-2-yl)-2,3-dihydro-1H-imidazo[1,2-a]imidazol-5-yl)phenyl)-3-(4-(2-hydroxyethyl)piperazin-1-yl)propanamide;
N-(2-fluoro-5-(6-(6-methylpyridin-2-yl)-2,3-dihydro-1H-imidazo[1,2-a]imidazol-5-yl)phenyl)-3-(4-(2,2,2-trifluoroethyl)piperazin-1-yl)propanamide;
2-(Dimethylamino)-N-(2-fluoro-5-(6-(6-methylpyridin-2-yl)-2,3-dihydro-1H-imidazo[1,2-a]imidazol-5-yl)phenyl)acetamide;
N-(2-fluoro-5-(6-(6-methylpyridin-2-yl)-2,3-dihydro-1H-imidazo[1,2-a]imidazol-5-yl)phenyl)-2-(4-methylpiperazin-1-yl)acetamide;
N-(2-fluoro-5-(6-(6-methylpyridin-2-yl)-2,3-dihydro-1H-imidazo[1,2-a]imidazol-5-yl)phenyl)-4-(4-methylpiperazin-1-yl)butanamide;
N-(5-(6-(5-chloro-2-fluorophenyl)-2,3-dihydro-1H-imidazo[1,2-a]imidazol-5-yl)-2-fluorophenyl)-3-(4-methylpiperazin-1-yl)propanamide;
3-(Dimethylamino)-N-(2-fluoro-5-(6-(6-methylpyridin-2-yl)-2,3-dihydro-1H-imidazo[1,2-a]imidazol-5-yl)phenyl)propanamide;
N-(5-(6-(5-chloro-2-fluorophenyl)-2,3-dihydro-1H-imidazo[1,2-a]imidazol-5-yl)-2-fluorophenyl)-3-(piperidin-1-yl)propanamide;
N-(2-fluoro-5-(6-(6-methylpyridin-2-yl)-2,3-dihydro-1H-imidazo[1,2-a]imidazol-5-yl)phenyl)-4-(piperidin-1-yl)butanamide; and
1-(4-((2-Fluoro-5-(6-(6-methylpyridin-2-yl)-2,3-dihydro-1H-imidazo[1,2-a]imidazol-5-yl)phenyl)amino)-4-oxobutyl)-1-methylpiperidin-1-ium chloride.

4: The compound of formula (I) or salt thereof according to claim 1, wherein R₁ is pyridyl optionally substituted by one or more methyl, and R₂ is selected from the group consisting of NH-heterocycloalkyl, wherein said heterocycloalkyl is optionally substituted by one or more —(C₁-C₆)alkyl; —NH—(C₁-C₆)alkylene-heterocycloalkyl, wherein said heterocycloalkyl is optionally substituted by one or more —(C₁-C₆)alkyl; and —NH—C(O)—(C₁-C₆)alkylene-heterocycloalkyl, wherein said heterocycloalkyl is optionally substituted by one or more —(C₁-C₆)alkyl.

5: The compound of formula (I) or salt thereof according to claim 1, wherein R₁ is 6-methylpyridin-2-yl and R₂ is selected from the group consisting of -1-isopropylpiperidin-4-amine and -(1-methylpiperidin-4-yl)methanamine.

6: The compound of formula (I) or salt thereof according to claim 5, selected from the group consisting of:
N-(2-fluoro-5-(6-(6-methylpyridin-2-yl)-2,3-dihydro-1H-imidazo[1,2-a]imidazol-5-yl)phenyl)-1-isopropylpiperidin-4-amine; and
2-Fluoro-N-((1-methylpiperidin-4-yl)methyl)-5-(6-(6-methylpyridin-2-yl)-2,3-dihydro-1H-imidazo[1,2-a]imidazol-5-yl)aniline.

7: A pharmaceutical composition comprising the compound of formula (I) or salt thereof according to claim 1, in admixture with one or more pharmaceutically acceptable carriers or excipients.

8: The pharmaceutical composition according to claim 7, which is formulated for administration by inhalation.

9. (canceled)

10: A method of treating a disease, disorder or condition mediated by ALK5 signaling pathway in a mammal, comprising administering the compound of formula (I) or salt thereof according to claim 1 to a subject in need thereof.

11: A method of treating fibrosis and/or a disease disorder or condition that involves fibrosis, comprising administering the compound of formula (I) or salt thereof according to claim 1 to a subject in need thereof.

12: The method according to claim 11, wherein the fibrosis is at least one selected from the group consisting of pulmonary fibrosis, idiopathic pulmonary fibrosis (IPF), hepatic fibrosis, renal fibrosis, ocular fibrosis, cardiac fibrosis, arterial fibrosis and systemic sclerosis.

13: The method according to claim 12, wherein the fibrosis comprises idiopathic pulmonary fibrosis (IPF).
